(19) United States
(12) Patent Application Publication
(10) Pub. No.: US 2025/0195501 A1
(43) Pub. Date: Jun. 19, 2025

(54) CELL ENTRY-MODULATING AGENTS AND USES THEREFOR

(71) Applicant: Griffith University, Nathan (AU)

(72) Inventors: Michael Paul Jennings, Nathan (AU); Christopher James Day, Nathan (AU); Milton Kiefel, Nathan (AU); Thomas Haselhorst, Nathan (AU)

(21) Appl. No.: 17/998,099

(22) PCT Filed: May 7, 2021
(86) PCT No.: PCT/AU2021/050422

§ 371 Date: Nov. 7, 2022

(30) Foreign Application Priority Data
May 7, 2020 (AU) 2020901462

(51) Int. Cl. A61K 31/4745; A61K 39/00; C07K 14/005

(52) U.S. Cl. CPC A61K 31/4745; C07K 14/005; A61K 39/00; C12N 2770/20022; C12N 2770/20034

(57) ABSTRACT

The present disclosure relates generally to the use of compounds that inhibit coronavirus entry into an angiotensin converting enzyme 2 (ACE2)-expressing cell, in methods and compositions for inhibiting the interaction of coronavirus to an ACE2-expressing cell and for treating or inhibiting the development of coronavirus infections.

### CROSS-REFERENCE TO RELATED APPLICATIONS

This application claims priority to Australian Provisional Application No. 2020901462 entitled “Cell entry-modulating agents and uses therefor” filed 7 May 2020, the contents of which are incorporated herein by reference in their entirety.

### FIELD

This disclosure relates generally to the use of compounds that inhibit coronavirus entry into an angiotensin converting enzyme 2 (ACE2)-expressing cell, in methods and compositions for inhibiting the interaction of coronavirus to an ACE2-expressing cell and for treating or inhibiting the development of coronavirus infections.

### BACKGROUND

The coronavirus SARS-CoV-2 is a recently emerged virus that causes an often-fatal respiratory disease, COVID-19. The current pandemic caused by SARS-CoV-2 is a health emergency that requires the development of new vaccines and drugs to prevent or treat this disease. Most antiviral drug strategies target viral proteins, or host factors required for intracellular replicative processes. Inhibiting viral entry into host cells via blocking access to cell surface viral receptors can also be a successful strategy; the best example being the entry inhibitor drug Maraviroc, which binds to the HIV1 co-receptor CCR5 to block infection (Dorr et al., Antimicrob Agents Chemother 2005, 49 (11), 4721-32).

SARS-CoV-2 is closely related to severe acute respiratory syndrome coronavirus (SARS-CoV) (Lu et al., Lancet 2020, 395 (10224), 565-574) and recent studies have demonstrated that SARS-CoV-2 spike protein, like SARS-CoV, uses the angiotensin converting enzyme 2 (ACE2) as a cellular receptor to initiate infection (Hoffmann et al., Cell 2020 181(2): 271-280). SARS-CoV-2 engages the ACE2 receptor with higher affinity binding than SARS-CoV (Wrapp et al., Science 2020, 367 (6483), 1260-1263). Repurposing existing drugs is the most rapid path to clinical intervention for emerging diseases. In a recent study we used a high-throughput SPR assay to screen compound libraries and identified registered drugs that bind to human complement receptor 3 as a host-receptor-blocking strategy to prevent bacterial infection (Poole et al., mBio 2020, 11 (2)).

### SUMMARY

The present disclosure is predicated in part on the identification of compounds that inhibit the interaction between ACE2 and the coronavirus SARS-CoV-2 spike protein receptor binding domain protein (RBD), which may serve inter alia as coronavirus entry inhibitors. In specific embodiments, peptide compounds that mimic ACE2-interacting structural determinants of a coronavirus spike protein RBD are disclosed, which not only serve as competitive inhibitors of coronavirus entry into ACE2 polypeptide-expressing cells, but also serve as immune-modulating agents for eliciting an immune response to a coronavirus, as coronavirus detection agents for detecting the presence of coronavirus in subjects, and as affinity agents for isolating antigen-binding molecules that bind specifically to a coronavirus.

Accordingly, disclosed herein in one aspect are methods for inhibiting interaction of a coronavirus with an ACE2 polypeptide-expressing cell. These methods generally comprise, consist or consist essentially of contacting the cell with an ACE2 polypeptide-interacting compound that inhibits binding of an ACE2-interacting coronavirus (e.g., SARS-CoV-2) spike protein RBD or structural mimetic thereof to the ACE2 polypeptide, thereby inhibiting the interaction of the coronavirus with the cell. In specific embodiments, the ACE2 polypeptide-interacting compound is selected from the compounds listed in TABLE 1.

In specific embodiments, the ACE2-interacting coronavirus (e.g., SARS-CoV-2) spike protein RBD structural mimetic is a peptide that comprises, consists or consists essentially of the amino acid sequence set forth in SEQ ID NO: 1 or 2, or an amino acid sequence corresponding thereto.

The interaction may include one or both of binding of the coronavirus to the cell and entry of the coronavirus into the cell. The cell may be a lung cell (e.g., an alveolar cell), an enterocyte, an endothelial cell, an epithelial cell (e.g., a nasal or nasopharyngeal epithelial cell), a kidney cell (e.g., brush border of proximal tubular epithelial cells) or an arterial smooth muscle cell. In specific embodiments, the ACE2-expressing cell is a cell of the respiratory tract.

Disclosed herein in another aspect are methods for treating or inhibiting the development of a coronavirus infection in a subject, wherein the coronavirus interacts with an ACE2 polypeptide-expressing cell. These methods generally comprise, consist or consist essentially of administering to the subject an effective amount of an ACE2 polypeptide-interacting compound that inhibits binding of an ACE2-interacting coronavirus (e.g., SARS-CoV-2) spike protein RBD or structural mimetic thereof to the ACE2 polypeptide, to thereby treat or inhibit the development of the coronavirus infection in the subject. In specific embodiments, the ACE2 polypeptide-interacting compound is selected from the compounds listed in TABLE 1. Suitably, the effective amount is one that inhibits transmission of the virus to the subject and/or spreading of the virus within the subject.

In any of the aspects disclosed herein, the coronavirus is suitably selected from Severe Acute Respiratory Syndrome virus (SARS-CoV), Severe Acute Respiratory Syndrome virus 2 (SARS-CoV-2) and Middle East Respiratory Syndrome virus (MERS).

In any of the aspects disclosed herein, the ACE2 polypeptide-interacting compound may be formulated for oral delivery, for systemic delivery or topical delivery. In some embodiments, the ACE2 polypeptide-interacting compound is formulated for delivery to the respiratory tract.

In any of the aspects disclosed herein, the ACE2 polypeptide-interacting compound may be administered concurrently with an ancillary agent (e.g., an antimicrobial agent).

In related aspects, the present disclosure provides compositions for use in therapy or prophylaxis of a coronavirus infection in a subject, wherein the coronavirus interacts with an ACE2 polypeptide-expressing cell. These compositions generally comprise, consist or consist essentially of an ACE2 polypeptide-interacting compound that inhibits binding of an ACE2-interacting coronavirus (e.g., SARS-CoV-2) spike protein RBD or structural mimetic thereof to the ACE2 polypeptide, and optionally a pharmaceutically acceptable carrier. In specific embodiments, the ACE2 polypeptide-interacting compound is selected from the compounds listed in TABLE 1.

Disclosed herein are ACE2 polypeptide-interacting peptide compounds that comprise, consist or consist essentially of the amino acid sequence set forth in SEQ ID NO: 1 or 2, or an amino acid sequence corresponding thereto, which mimic structural determinants of an ACE2-interacting coronavirus spike protein RBD. Accordingly, in another aspect, the present disclosure provides a peptide compound that comprises, consists or consists essentially of the amino acid sequence set forth in SEQ ID NO: 1 or 2, or an amino acid sequence corresponding thereto.

In a related aspect, disclosed herein is a polynucleotide comprising, consisting or consisting essentially of a coding sequence for a peptide compound that comprises, consists or consists essentially of the amino acid sequence set forth in SEQ ID NO: 1 or 2, or an amino acid sequence corresponding thereto.

In another related aspect, the present disclosure provides a nucleic acid construct comprising a polynucleotide comprising, consisting or consisting essentially of a coding sequence for a peptide compound that comprises, consists or consists essentially of the amino acid sequence set forth in SEQ ID NO: 1 or 2, or an amino acid sequence corresponding thereto, wherein the polynucleotide is operably connected to a regulatory element (e.g., a promoter).

In a further related aspect, the present disclosure provides a host cell comprising the nucleic acid construct broadly described above.

In accordance with the present disclosure, these peptide compounds are disclosed as immune-modulating agents for eliciting an immune response to an ACE2-interacting coronavirus, as coronavirus detection agents for detecting the presence of coronavirus in subjects, and as affinity agents for isolating antigen-binding molecules that bind specifically to a coronavirus. Therefore, in another aspect, the present disclosure encompasses methods of eliciting an immune response to an ACE2-interacting coronavirus in a subject. These methods generally comprise, consist or consist essentially of immunizing the subject with a peptide that comprises, consists or consists essentially of the amino acid sequence set forth in SEQ ID NO: 1 or 2, or an amino acid sequence corresponding thereto, to thereby elicit an immune response to the ACE2-interacting coronavirus in the subject.

Also disclosed herein in another aspect are methods of producing an antigen-binding molecule (e.g., a neutralizing antigen-binding molecule) that binds specifically with a coronavirus spike protein that interacts with an ACE2 polypeptide-expressing cell. These methods generally comprise: (1) immunizing an animal with a peptide that comprises, consists or consists essentially of the amino acid sequence set forth in SEQ ID NO: 1 or 2, or an amino acid sequence corresponding thereto; (2) detecting a B cell from the animal, which binds specifically with the coronavirus spike protein; and (3) isolating the antigen-binding molecule expressed by that B cell.

In a related aspect, methods are disclosed herein for producing an antigen-binding molecule that binds specifically with a coronavirus spike protein that interacts with an ACE2 polypeptide-expressing cell. These methods generally comprise: (1) screening a library of antigen-binding molecules with a peptide that comprises, consists or consists essentially of the amino acid sequence set forth in SEQ ID NO: 1 or 2, or an amino acid sequence corresponding thereto; (2) detecting an antigen-binding molecule that binds specifically with the peptide; and (3) isolating the detected antigen-binding molecule.

Also disclosed herein in a further aspect are antigen-binding molecules produced by the immunizing or screening methods disclosed herein, or a derivative antigen-binding molecule with the same epitope-binding specificity as the antigen-binding molecule. The derivative antigen-binding molecule may be selected from antibody or ‘antigen-binding’ fragments (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen binding/recognition site. Also encompassed are cells including hybridomas and cell lines that are capable of producing the antigen-binding molecules of the disclosure, and methods of producing antigen-binding molecules from those cells. In specific embodiments, the antigen-binding molecules produced by the methods and cells of the disclosure are preferably neutralizing antigen-binding molecules.

Disclosed herein in a related aspect is an immune-modulating composition comprising an antigen-binding molecule produced by methods disclosed herein, and a pharmaceutically acceptable carrier, diluent or adjuvant.

In a related aspect, the present disclosure contemplates methods for treating an ACE2-interacting coronavirus infection in a subject. These methods generally comprise administering to the subject an effective amount of an antigen-binding molecule produced by methods disclosed herein.

In a related aspect, disclosed herein are kits for treating an ACE2-interacting coronavirus infection in a subject. These kits generally comprise: an antigen-binding molecule that binds specifically to a coronavirus that interacts with an ACE2 polypeptide-expressing cell, and optionally instructional material for performing the treatment.

In yet another aspect, disclosed herein are methods for detecting the presence of a coronavirus infection in a subject, wherein the coronavirus interacts with an ACE2 polypeptide-expressing cell. These methods generally comprise contacting a biological sample with an antigen-binding molecule produced by methods disclosed herein and detecting the presence of an immune complex comprising the antigen-binding molecule and the coronavirus or component thereof (e.g., spike protein or spike protein RBD) in the sample, thereby detecting the presence of the coronavirus infection.

Disclosed herein in a further aspect are methods for detecting the presence of an immune response (e.g., a humoral immune response) to a coronavirus infection in a subject, wherein the coronavirus interacts with an ACE2 polypeptide-expressing cell. These methods generally comprise contacting a biological sample with a peptide that comprises, consists or consists essentially of the amino acid sequence set forth in SEQ ID NO: 1 or 2, or an amino acid sequence corresponding thereto, and detecting the presence of an immune complex comprising the peptide and an antigen-binding molecule in the sample, thereby detecting the presence of an immune response to the coronavirus infection in the subject.

In any of the above aspects, the biological sample is suitably selected from tissue and fluid samples, representative examples of which include: biological fluids and/or tissues obtained or derived from the respiratory tract including mouth, nose, throat and lungs.

Also disclosed herein are kits for detecting the presence of an ACE2-interacting coronavirus infection in a subject, or for detecting the presence of an immune response to an ACE2-interacting coronavirus infection in a subject. These kits generally comprise: an antigen-binding molecule that binds specifically to a coronavirus that interacts with an ACE2 polypeptide-expressing cell and/or a peptide that comprises, consists or consists essentially of the amino acid sequence set forth in SEQ ID NO: 1 or 2, or an amino acid sequence corresponding thereto. Suitably, the kits of the present disclosure further comprise instructional material for performing any one or more of the detection methods disclosed herein.

In any of the above aspects, the antigen-binding molecule and/or the peptide are detectably labeled.

In accordance with the present disclosure, the ACE2 polypeptide-interacting compound is preferably selected from the compounds listed in TABLE 1.

TABLE 1

| | |
|---|---|
| 1. A compound of formula (I): | |
| 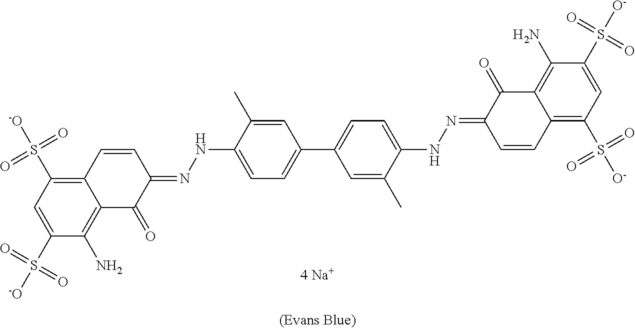 | (I) |
| or derivative or prodrug or pharmaceutically acceptable salt thereof. | |
| 2. A compound of formula (IIa): | |
| 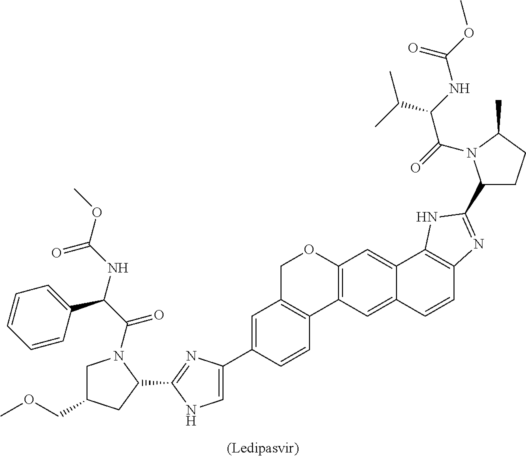 | (IIa) |
| or a derivative or prodrug or pharmaceutically acceptable salt thereof. | |
| 3. A compound of formula (IIIa): | |
| 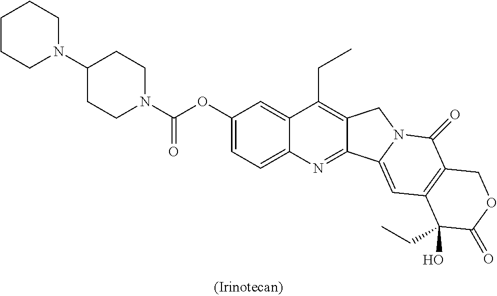 [SMILES: CCc1c2c(nc3ccc(OC(=O)N4CCC(N5CCCCC5)CC4)cc13)-c1cc3c(c(=O)n1C2)COC(=O)[C@]3(O)CC] | (IIIa) |
| or derivative or prodrug or pharmaceutically acceptable salt thereof. | |
| 4. A compound of formula (IIIb): | |
| [symbol] | (IIIb) |
| wherein: | |
| R¹ is selected from carbon and heteroatoms, preferably nitrogen; and | |
| R² is selected from hydrogen and C₁₋₆ alkyl, preferably C₁₋₃ alkyl, | |
| or a derivative or pharmaceutically acceptable salt thereof. | |
| 5. A compound of formula (IIIb1): | |
| 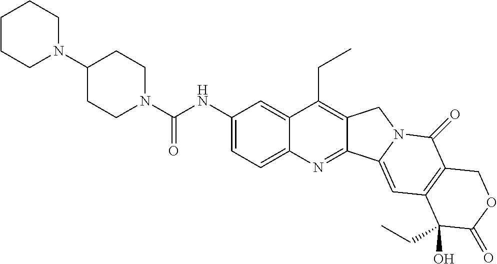 [SMILES: CCc1c2c(nc3ccc(NC(=O)N4CCC(N5CCCCC5)CC4)cc13)-c1cc3c(c(=O)n1C2)COC(=O)[C@]3(O)CC] | (IIIb1) |
| or a derivative or prodrug or pharmaceutically acceptable salt thereof. | |
| 6. A compound of formula (IIIb2): | |
| 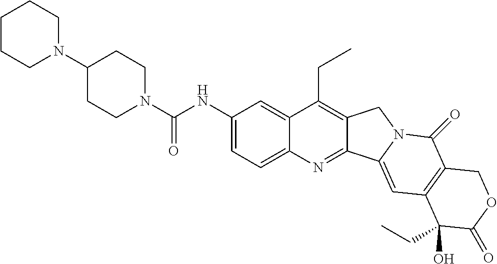 [SMILES: CCc1c2c(nc3ccc(NC(=O)N4CCC(N5CCCCC5)CC4)cc13)-c1cc3c(c(=O)n1C2)COC(=O)[C@]3(O)CC] | (IIIb2) |
| or derivative or prodrug or pharmaceutically acceptable salt thereof. | |
| 7. A compound of formula (IIIb3): | |
| 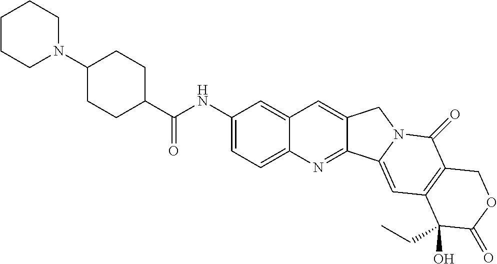 [SMILES: CC[C@@]1(O)C(=O)OCc2c1cc1n(c2=O)Cc2cc3cc(NC(=O)C4CCC(N5CCCCC5)CC4)ccc3nc2-1] | (IIIb3) |
| or derivative or prodrug or pharmaceutically acceptable salt thereof. | |
| 8. A compound of formula (IIIb4): | |
| 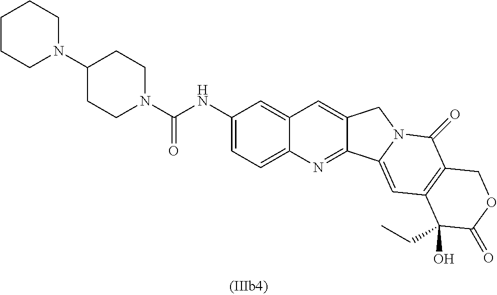 [SMILES: CC[C@@]1(O)C(=O)OCc2c1cc1n(c2=O)Cc2cc3cc(NC(=O)N4CCC(N5CCCCC5)CC4)ccc3nc2-1] | (IIIb4) |
| or a derivative or prodrug or pharmaceutically acceptable salt thereof. | |
| 9. A compound of formula (IVa): | |
| 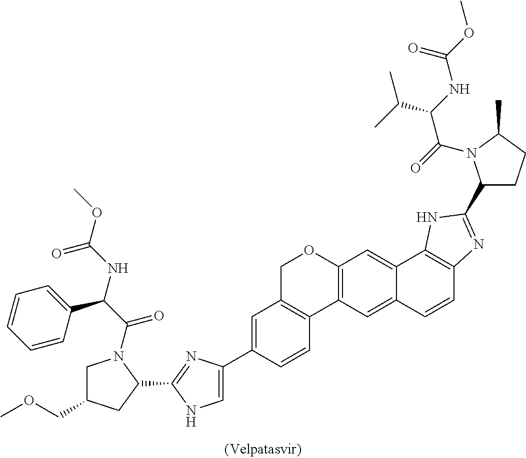 | (IVa) |
| or a derivative or prodrug or pharmaceutically acceptable salt thereof | |
| 10. A compound of formula (V): | |
| 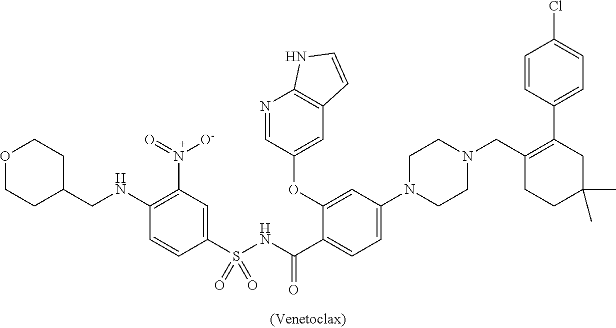 | (V) |
| or derivative or prodrug or pharmaceutically acceptable salt thereof | |
| 11. A peptide comprising, consisting or consisting essentially of the amino acid sequence | |
| NCYFPLQSYGFQPTNGV [SEQ ID NO: 1] or NCYFPLQSYGFQPTNGVGY [SEQ ID NO: 2], or | |
| an amino acid sequence corresponding thereto. | |

[symbol]

indicates data missing or illegible when filed

Also disclosed herein in still another aspect are novel ACE2 polypeptide-interacting compounds that are Irinotecan derivatives represented by formula (IIIb):

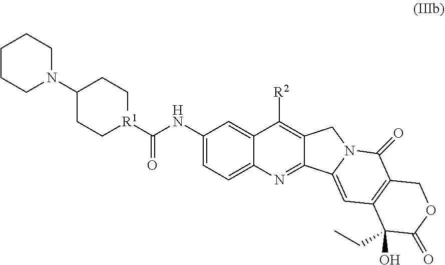

wherein:
R¹ is selected from carbon and heteroatoms, preferably nitrogen; and
R² is selected from hydrogen and C₁₋₆ alkyl, preferably C₁₋₃ alkyl,
or a derivative or pharmaceutically acceptable salt thereof.

In specific embodiments, these compounds are selected from:

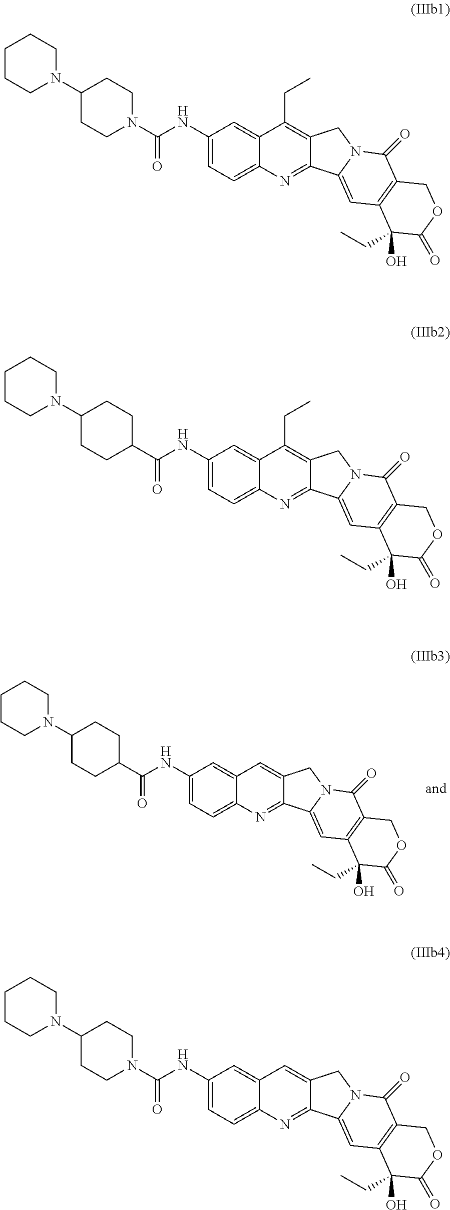 [SMILES: CCc1c2c(nc3ccc(NC(=O)N4CCC(N5CCCCC5)CC4)cc13)-c1cc3c(c(=O)n1C2)COC(=O)[C@]3(O)CC ; CCc1c2c(nc3ccc(NC(=O)C4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2cc3cc(NC(=O)C4CCC(N5CCCCC5)CC4)ccc3nc2-1 ; CC[C@@]1(O)C(=O)OCc2c1cc1n(c2=O)Cc2cc3cc(NC(=O)N4CCC(N5CCCCC5)CC4)ccc3nc2-1]

or a derivative or pharmaceutically acceptable salt thereof.

Yet another aspect of the present disclosure provides methods of identifying an agent that inhibits interaction of an ACE2-interacting coronavirus with an ACE2 polypeptide-expressing cell, or that inhibits entry of an ACE2-interacting coronavirus into an ACE2 polypeptide-expressing cell, or that is useful for treating or inhibiting the development of an ACE2-interacting coronavirus infection. These methods generally comprise: contacting an ACE2 polypeptide or an ACE2 polypeptide-expressing cell with a candidate agent in the presence of a peptide that comprises, consists or consists essentially of the amino acid sequence set forth in SEQ ID NO: 1 or 2, or an amino acid sequence corresponding thereto; and detecting whether the candidate agent inhibits binding of the peptide with the ACE2 polypeptide or ACE2 polypeptide-expressing cell, which indicates that the candidate agent is an agent that inhibits interaction of an ACE2-interacting coronavirus with an ACE2 polypeptide-expressing cell, or that inhibits entry of an ACE2-interacting coronavirus into an ACE2 polypeptide-expressing cell, or that is useful for treating or inhibiting the development of an ACE2-interacting coronavirus infection.

### BRIEF DESCRIPTION OF THE DRAWINGS

FIG. 1 is an illustration showing structure of the human ACE2-S1 domain (RBD) of the SARS-CoV spike protein initially used to select regions for molecular docking screen. The structure was determined by (PDB 2AJF) 6. A. SARS-CoV S spike protein bound to ACE2 (PDB 2AJF) with marked ACE2 active site and SARS-CoV S protein interacting site. B. For molecular docking screening experiments SARS-CoV S spike protein was removed and a rectangular box (50 Å×60 Å×40 Å) was centered around HIS-34 of the ACE2 coordinates.

FIG. 2 is a graphical representation showing Evans Blue identification in SPR screen. Highlighted spot is Evans blue (CA sample #: SN01005402, molecular weight 962 g/mol).

FIG. 3 is a graphical representation showing Evans Blue identification in SPR screen. Sensorgram of different concentrations of Evans Blue with ACE2.

FIG. 4 is a graphical representation showing competition SPR analysis of Evans Blue against the RBD-1 peptide. Peptide alone, compound alone and the competition. Percentage competition is calculated by the reduction in final peptide binding divided by the difference between the peptide and the compound alone.

FIG. 5 is an illustration showing a molecular representation of the interactions of Evans Blue with the key, SARSCoV-2 RBD-interacting residues of human ACE2 that are marked in red; compound is also shown.

FIG. 6 is an illustration showing a molecular representation of the interactions of Irinotecan with the key, SARSCoV-2 RBD-interacting residues of human ACE2 that are marked in red; compound is also shown.

FIG. 7 is an illustration showing a molecular representation of the interactions of Velpatasvir with the key, SARSCoV-2 RBD-interacting residues of human ACE2 that are marked in red; compound is also shown.

FIG. 8 is an illustration showing a molecular representation of the interactions of Venetoclax with the key, SARSCoV-2 RBD-interacting residues of human ACE2 that are marked in red; compound is also shown.

FIG. 9 is an illustration showing a molecular representation of the interactions of Ledipasvir with the key, SARSCoV-2 RBD-interacting residues of human ACE2 that are marked in red; compound is also shown.

FIG. 10 is a graphical representation showing competition SPR analysis of Irinotecan against the RBD-1 peptide. Peptide alone, compound alone and the competition. Percentage competition is calculated by the reduction in final peptide binding divided by the difference between the peptide and the compound alone.

FIG. 11 is a graphical representation showing competition SPR analysis of Velpatasvir against the RBD-1 peptide. Peptide alone, compound alone and the competition. Percentage competition is calculated by the reduction in final peptide binding divided by the difference between the peptide and the compound alone.

FIG. 12 is a graphical representation showing competition SPR analysis of Venetoclax against the RBD-1 peptide. Peptide alone, compound alone and the competition. Percentage competition is calculated by the reduction in final peptide binding divided by the difference between the peptide and the compound alone.

FIG. 13 is a graphical representation showing competition SPR analysis of Ledipasvir against the RBD-1 peptide. Peptide alone, compound alone and the competition. Percentage competition is calculated by the reduction in final peptide binding divided by the difference between the peptide and the compound alone.

FIG. 14 is an illustration showing a molecular representation of the interactions of SN-38 with the key, SARSCoV-2 RBD-interacting residues of human ACE2 that are marked in red; compound is also shown.

FIG. 15 is an illustration showing a molecular representation of the interactions of Irinotecan with the key, SARSCoV-2 RBD-interacting residues of human ACE2 that are marked in red; compound is also shown.

FIG. 16 is an illustration showing a molecular representation of the interactions of an Irinotecan derivative, in which the carbamate ester linker of Irinotecan is substituted with an amide linker, with the key, SARSCoV-2 RBD-interacting residues of human ACE2 that are marked in red; compound is also shown.

FIG. 17 is an illustration showing a molecular representation of the interactions of an Irinotecan derivative, in which the carbamate ester linker of Irinotecan is substituted with a urea linker, with the key, SARSCoV-2 RBD-interacting residues of human ACE2 that are marked in red; compound is also shown.

Some figures and text contain color representations or entities. Color illustrations are available from the Applicant upon request or from an appropriate Patent Office. A fee may be imposed if obtained from a Patent Office.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The term “adjuvant” as used herein refers to a compound that, when used in combination with a specific immunogen (e.g., a peptide of the present disclosure that is a structural mimetic of an ACE2-interacting coronavirus spike protein RBD) in a composition, will augment the resultant immune response, including intensification or broadening the specificity of either or both antibody and cellular immune responses.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same composition. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells. As used herein, the terms “candidate agent” and “test agent” are used interchangeably to refer to agents and/or compositions that are to be screened for their ability to inhibit inhibits interaction of an ACE2-interacting coronavirus with an ACE2 polypeptide-expressing cell, or to inhibit entry of an ACE2-interacting coronavirus into an ACE2 polypeptide-expressing cell, or to treat or inhibit the development of an ACE2-interacting coronavirus infection.

As used herein, unless otherwise specified, the term “alkyl” includes saturated aliphatic groups, including straight-chain alkyl groups (e.g., methyl, ethyl, propyl, butyl, pentyl, hexyl, etc.), and branched-chain alkyl groups (isopropyl, tert-butyl, iso-butyl, sec-butyl, etc.). The expression “C_{x-y}alkyl”, wherein x is 1-2 and y is 2-6 indicates an alkyl group (straight- or branched-chain) containing the specified number of carbon atoms. For example, the term C₁₋₄alkyl includes methyl, ethyl, propyl, butyl, iso-propyl, tert-butyl, sec-butyl and iso-butyl. In some embodiments, a straight chain or branched chain alkyl has 4 or fewer carbon atoms (i.e., C₁₋₄). In some embodiments a straight chain or branched chain alkyl has 3 or fewer carbon atoms (i.e., C₁₋₃). Where indicated, an alkyl group may be substituted by one, two or three substituents. Non-limiting optional substituents for an alkyl group include halo; CF₃; OR^{a}; SR^{a}; NR^{a}R^{b}; and COR^{c}; wherein Ra and R^{b} are independently selected from hydrogen and C₁₋₄alkyl and R^{c} is C₁₋₄alkyl or R^{c} is phenyl optionally substituted with one, two or three substituents selected from halogen, CF₃, OH or OC₁₋₄alkyl.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, unless otherwise specified, the term “alkylene” includes saturated aliphatic linking groups, including straight-chain alkyl groups (e.g., methylene, ethylene, propylene, butylene etc.), and branched-chain alkyl groups (iso-propylene, tert-butylene, iso-butylene, sec-butylene). The expression “C_{x-y}alkylene”, wherein x is 1-2 and y is 2-4 indicates an alkylene group (straight- or branched-chain) containing the specified number of carbon atoms. For example, the term C₁₋₄alkylene includes methylene, ethylene, propylene, butylene, iso-propylene, tert-butylene, sec-butylene and iso-butylene. In some examples, a straight chain or branched chain alkylene has 4 or fewer carbon atoms (i.e., C₁₋₄). In some examples a straight chain or branched chain alkylene has 3 or fewer carbon atoms (i.e. C₁₋₃). In some preferred examples the alkylene linking group is propylene or sec-butylene.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present disclosure include polyclonal and monoclonal antibodies as well as their antigen-binding fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen-binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

The term “antimicrobial agent” as used herein refers to any agent with antimicrobial activity, i.e., the ability to inhibit or reduce the growth and/or kill a microbe, e.g., by at least about 30%, at least about 40%, at least about 50%, at least about 75%, at least about 90% or more, as compared to in the absence of an antimicrobial agent. The term “antimicrobial agent” encompasses agents that that inhibit or reduce the growth and/or kill a microbe by directly interacting with the microbe and/or cells of the host in which the microbe resides or is located. Non-limiting examples of antimicrobial agents include a silver nanoparticle, a small molecule, a peptide, a peptidomimetics, an antibody or a fragment thereof, a nucleic acid, an enzyme (e.g., an antimicrobial metalloendopeptidase such as lysostaphin), an aptamer, a drug, an antibiotic, a chemical or any entity that can inhibit the growth and/or kill a microbe. Examples of an antimicrobial peptide that can be included in the compositions described herein, include, but are not limited to, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium and/or xycitrin or modified versions or analogues thereof. Representative antimicrobial agents include, antibiotics, antifungals, antiprotozoals, antimalarials, antituberculotics and antivirals, and any mixtures thereof. In some embodiments, the antimicrobial agent is an antiviral agent, which encompasses agents that are effective for inhibiting the formation and/or replication of a virus in a mammal. This includes agents that interfere with either host or viral mechanisms necessary for the formation and/or replication of a virus in a mammal. Such agents can be selected from immunomodulatory agents, inhibitors of a virus polymerase or inhibitors of another target in the virus life cycle. Examples of anti-viral agents include a-methyl-1-adamantanemethylamine, hydroxy-ethoxymethylguanine, adamantanamine, 5-iodo-2′-deoxyuridine, trifluorothymidine, AZT, adenine arabinoside, Abacavir, Aciclovir, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Entry, inhibitors, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir, (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Stavudine, Tenofovir, Tenofovir, disoproxil, lipranavir, Trifluridine, Trizivir, Tromantadine, Truvada, Valaciclovir, (Valtrex), Valganciclovir, Vicriviroc, Vidarabine, Viramidine, Zalcitabine, Zanamivir, (Relenza), and Zidovudine.

The terms “angiotensin converting enzyme 2 polypeptide”, “ACE2 polypeptide” and “ACE2” are used interchangeably herein to refer to a polypeptide comprising the amino acid sequence of a precursor ACE2 polypeptide (e.g., the human precursor ACE2 polypeptide set forth in GenBank Accession NP_001358344), or processed form thereof (e.g., a mature ACE2 polypeptide in which the signal peptide, as defined for example by amino acids 1-17, has been removed and/or a carboxy terminal portion, as defined for example by amino acids 709-805, has been removed), or biologically active fragments of a precursor or processed form an ACE2 polypeptide, as well as allelic variants of any of these. ACE2 polypeptides lower blood pressure by catalyzing the hydrolysis of angiotensin II (Ang II; a vasoconstrictor peptide) into angiotensin 1-7 (Ang 1-7; a vasodilator). ACE2 counters the activity of the related angiotensin-converting enzyme (ACE) by reducing the amount of Ang II and increasing the amount of angiotensin. Also, degree and location of glycosylation or other post-translation modifications may vary depending on the chosen host and the nature of the hosts cellular environment. The term “ACE2 polypeptide” is also intended to encompass ACE2 polypeptides that have either been chemically modified relative to a reference ACE2 polypeptide and/or contain one or more amino acid sequence alterations relative to a reference ACE2 polypeptide and/or contain truncated amino acid sequences relative to a reference full-length or processed ACE2 polypeptide. The term “ACE2 polypeptide” also encompasses proteinaceous molecules with a slightly modified amino acid sequence, for instance, polypeptides having a modified N-terminal end including N-terminal amino acid deletions or additions, and/or polypeptides that have been chemically modified relative to a reference ACE2 polypeptide. ACE2 polypeptides also encompass proteinaceous molecules exhibiting substantially the same or better bioactivity than a reference ACE2 polypeptide, or, alternatively, exhibiting substantially modified or reduced bioactivity relative to a reference or ACE2 polypeptide. They also include, without limitation, polypeptides having an amino acid sequence that differs from the sequence of a reference ACE2 polypeptide by insertion, deletion, or substitution of one or more amino acids and in illustrative examples, encompass proteinaceous molecules that exhibit at least about 10%, 20%, 30%, 40%, 50%, 60%, 70%, 80%, 90%, 100%, 110%, 120%, and 130% of the specific activity of a reference ACE2 polypeptide that has been produced in the same cell or cell type. The term “reference ACE2 polypeptide” refers to an ACE2 polypeptide that may be naturally-occurring or non-naturally-occurring, and includes within its scope a parent ACE2 polypeptide from which an ACE2 polypeptide is derived or corresponds thereto.

As used herein, the term “ACE2 polypeptide-expressing cell” refers to a cell that expresses an ACE2 polypeptide typically on its surface. Representative cells that express an ACE2 polypeptide include myeloid cells of the lungs, arteries, heart, kidney, and intestines. The ACE2-expressing cell is suitably selected from a lung cell (e.g., alveolar cell), an enterocyte, an endothelial cell and an arterial smooth muscle cell. In specific embodiments, the ACE2-expressing cell is a cell of the respiratory tract (e.g., a lung cell such as an alveolar cell).

The term “antagonist” as used herein refers to a molecule that partially or completely inhibits, by any mechanism, an effect of another molecule such as a receptor or intracellular mediator. In the context of the present invention, the term “antagonist” refers to a molecule that partially or completely inhibits an ACE2-interacting coronavirus, including its spike protein RBD, from interacting or forming a complex with an ACE. In specific embodiments, the antagonist is a direct antagonist that binds to or otherwise interacts with an ACE2 polypeptide or and ACE2 polypeptide-expressing cell.

As use herein, the term “binds”, “specifically binds to” or is “specific for” refers to measurable and reproducible interactions such as binding between a target and an antigen-binding molecule, which is determinative of the presence of the target in the presence of a heterogeneous population of molecules including biological molecules. For example, an antigen-binding molecule that binds to or specifically binds to a target (which can be an epitope) is an antigen-binding molecule that binds this target with greater affinity, avidity, more readily, and/or with greater duration than it binds to other targets. In one embodiment, the extent of binding of an antigen-binding molecule to an unrelated target is less than about 10% of the binding of the antibody to the target as measured, e.g., by a radioimmunoassay (RIA). In certain embodiments, an antigen-binding molecule that specifically binds to a target has a dissociation constant (Kd) of ≤1 μM, ≤100 nM, ≤10 nM, ≤1 nM, or ≤0.1 nM.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, sweat, nasal washes, nasal aspirate, breath, urine, semen, saliva, meningeal fluid, amniotic fluid, glandular fluid, lymph fluid, bronchial aspirate, synovial fluid, joint aspirate, cells, a cellular extract, secretions of the gastrointestinal tract, ascitic fluid, pleural fluid, intraocular fluid, fluid from a hydrocele (e.g. of the testis), fluid from a cyst, pancreatic secretions, intestinal secretions, aspiration fluids from breast and thyroid, etc. and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample (e.g., a sample of a tissue that associates with a cancer), or a tissue biopsy (e.g., a biopsy of a tissue that associates with a cancer), for example. The term “biological sample” also includes materials derived from a tissue culture or a cell culture. Any suitable methods for obtaining a biological sample can be employed; exemplary methods include, e.g., peeling cells using adhesive tape, scraping, phlebotomy, swab (e.g., buccal swab), biopsy touch preparations and fine needle aspirate biopsy procedure. Exemplary tissues include lymph node, esophagus, lung, lung washes, BAL (bronchoalveolar lavage), thyroid, skin, breast, ovary, endometrium, uterus, pancreas, spleen, thymus, bone marrow, colon, stomach, bladder, brain, salivary gland, prostate, testicles and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. In specific embodiments, the biological sample is a biological fluid and/or tissue obtained or derived from the respiratory tract including mouth, nose, throat and lungs.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). As used herein the term “complex”, unless described as otherwise, refers to the assemblage of two or more molecules (e.g., peptides, polypeptides or a combination thereof). In specific embodiments, the term “complex” refers to the assemblage of two or three polypeptides.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “compound library” as used herein refers to any collection of compounds, which includes a plurality of molecules of different structure. Compound libraries may include combinatorial chemical libraries or natural products libraries. Any type of molecule that is capable of interacting, binding or has affinity for a chimeric polypeptide or complex of the present invention, through interactions inclusive of non-covalent interactions, such as, for example, through hydrogen bonds, ionic bonds, van der Waals attractions, or hydrophobic interactions, may be present in the compound library. For example, compound libraries encompasses by this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A “conservative amino acid substitution” is one in which the amino acid residue is replaced with an amino acid residue having a similar side chain. Families of amino acid residues having similar side chains have been defined in the art, which can be generally sub-classified as follows:

TABLE 2
AMINO ACID SUB-CLASSIFICATION

| SUB-CLASSES | AMINO ACIDS |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, |
| | Lysine, Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 3 under the heading of exemplary and preferred substitutions. Amino acid substitutions falling within the scope of the present disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 3
EXEMPLARY AND PREFERRED
AMINO ACID SUBSTITUTIONS

| ORIGINAL | EXEMPLARY | PREFERRED |
|---|---|---|
| RESIDUE | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include regulatory elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

By “corresponds to” or “corresponding to” is meant an amino acid sequence that displays substantial sequence similarity or identity to a reference amino acid sequence. The amino acid sequence will preferably display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of coronavirus infections include acute febrile illness, malaise, fatigue, headache, flushing, diarrhea, nausea, vomiting, coughing including dry coughing, sore throat, runny nose, nasal congestion, and, in severe disease, symptoms of systemic inflammatory response syndrome including production of pro-inflammatory mediators, vascular leakage and organ failure.

“Entry inhibitors” are a particular class of drugs that inhibit the ability of a pathogen such a virus to successfully enter and thereby infect a target cell.

The term “expression” refers to the cellular processes involved in producing RNA and proteins and as appropriate, secreting proteins, including where applicable, but not limited to, for example, transcription, transcript processing, translation and protein folding, modification and processing.

As used herein, the term “halogen” includes fluorine, bromine, chlorine and iodine. Similarly, the term “halo” includes fluoro, chloro, bromo and iodo. In some examples, halo is preferably chloro.

As used herein, unless otherwise stated, the term “heterocycloalkyl” refers to saturated cyclic aliphatic groups containing 3- to 8-members including at least one endocyclic N atom, and optionally further including one or two further heteroatoms wherein a heteroatom replaces an endocyclic carbon atom. Preferred heteroatoms are nitrogen, oxygen and sulfur. In some embodiments a heteroatom is nitrogen or oxygen. The heterocycloalkyl moiety may be monocyclic or it may be a fused or bridged ring system. Preferably the heterocycloalkyl moiety is monocyclic. Examples of heterocycloalkyl rings formed when R¹ and R² together with the nitrogen atom to which they are attached form a 3 to 8 membered ring, include pyrrolidinyl and piperidinyl. Further examples include aziridinyl, azetidinyl and azepinyl. In some embodiments, the heterocycloalkyl ring has 4 to 6 members, preferably 5 or 6 members. In addition to the N atom, the heterocyclic ring may include one or more additional endocyclic heteroatoms selected from O, S and N to replace a carbon atom, for example morpholinyl and piperazinyl. A piperazinyl ring may be substituted on an endocyclic C or N atom. Optional substituents for a heterocycloalkyl group include C₁₋₄alkyl optionally substituted by OR^{d}, SR^{d}, CF₃, NR^{d}R^{e} or halogen; wherein R^{d} and R^{e} are independently selected from hydrogen and C₁₋₄alkyl.

The term “host” refers to any organism, or cell thereof, whether eukaryotic or prokaryotic into which a construct of the disclosure can be introduced. In particular embodiments, the term “host” encompasses prokaryotes including bacteria, and eukaryotes including unicellular eukaryotes such as yeast and fungi as well as multicellular eukaryotes such as animals non-limiting examples of which include invertebrate animals (e.g., insects, cnidarians, echinoderms, nematodes, etc.); vertebrate animals (e.g., fish, amphibian, reptile, bird, mammal); and mammals (e.g., rodents, primates such as humans and non-human primates).

The term “interaction”, including its grammatical equivalents, when referring to an interaction between two molecules, refers to the physical contact of the molecules with one another. Generally, such an interaction results in an activity (which produces a biological effect) of one or both of said molecules. The physical contact typically requires binding or association of the molecules with one another and may involve the formation of an induced magnetic field or paramagnetic field, covalent bond formation, ionic interaction (such as, for example, as occurs in an ionic lattice), a hydrogen bond, or alternatively, a van der Waals interaction such as, for example, a dipole-dipole interaction, dipole-induced dipole interaction, induced dipole-induced dipole interaction, or a repulsive interaction, or any combination of the above forces of attraction.

The term “neutralizing antigen-binding molecule” refers to an antigen-binding molecule that binds to or interacts with a target molecule or ligand and prevents binding or association of the target antigen to a binding partner such as a receptor or substrate, thereby interrupting the biological response that otherwise would result from the interaction of the molecules. In the case of the instant disclosure a neutralizing antigen-binding molecule suitably associates with a metastable or pre-fusion form of an enveloped virus fusion protein and preferably interferes or reduces binding and/or fusion of the fusion protein to a cell membrane.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

As used herein, unless otherwise defined, the term “optionally substituted” refers to substitution of a hydrogen atom on a group, for example an alkyl, phenyl or heterocycloalkyl group, with a non-hydrogen moiety as detailed herein. Any substituted group may bear one, two, three, or more optional substituents. In some examples, a substituted group will have one substituent.

It is also to be understood that definitions given to the variables of the generic formulae described herein will result in molecular structures that are in agreement with standard organic chemistry definitions and atom valencies.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the present disclosure include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (e.g., snakes, frogs, lizards etc.), and fish. In specific embodiments, the subject is a primate such as a human in need of treating or inhibiting the development of a coronavirus infection. However, it will be understood that the terms “patient,” “subject,” “host” or “individual” do not imply that symptoms are present.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

The term “pharmaceutically acceptable salt” as used herein is a broad term, and is to be given its ordinary and customary meaning to a person of ordinary skill in the art (and is not to be limited to a special or customized meaning), and refers without limitation to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

“Polypeptide”, “peptide”, “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

“Regulatory elements”, “regulatory sequences”, control elements”, “control sequences” and the like are used interchangeably herein to refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include enhancers, promoters, translation leader sequences, introns, Rep recognition element, intergenic regions and polyadenylation signal sequences. They include natural and synthetic sequences as well as sequences which may be a combination of synthetic and natural sequences.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, IIe, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

“Similarity” refers to the percentage number of amino acids that are identical or constitute conservative substitutions as defined in Tables 2 and 3 supra. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As used herein a “small molecule” refers to a compound that has a molecular weight of less than 3 kilodaltons (kDa), and typically less than 1.5 kilodaltons, and suitably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the disclosure to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, less than about 1 kDa or even less than about 0.5 kDa.

As used herein, the terms “treatment”, “treating”, and the like, refer to obtaining a desired pharmacologic and/or physiologic effect. The effect may be therapeutic in terms of a partial or complete cure for a disease or condition (e.g., a coronavirus infection) and/or adverse effect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

By “vector” is meant a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product (e.g., a polypeptide) is that which is most frequently observed in a population and is thus arbitrarily designed the “normal” or “wild-type” form of the gene or gene product.

Each embodiment described herein is to be applied mutatis mutandis to each and every embodiment unless specifically stated otherwise.

### 2. Compounds and Compositions for Modulating Coronavirus Interactions with ACE2 Polypeptide-Expressing Cells

The present disclosure is based in part on the identification of compounds that bind to ACE2 and that are able to inhibit binding of a coronavirus, including the spike protein RBD thereof, to ACE2 polypeptide-expressing cells and/or entry of the coronavirus into those cells. Thus, the compounds of the present disclosure have utility as coronavirus entry inhibitors for treating or inhibiting the development of coronavirus infections, and in certain peptide-based embodiments, as immune-modulating agents for eliciting an immune response to a coronavirus, as coronavirus detection agents for detecting the presence of coronavirus in subjects, and as affinity agents for isolating antigen-binding molecules that bind specifically to a coronavirus.

### 2.1 Inhibitors of Coronavirus ACE2 Polypeptide Interaction

The methods and compositions of the present disclosure feature compounds that bind to an ACE2 polypeptide and inhibit the interaction between ACE2 and an ACE2-interacting coronavirus (e.g., SARS-CoV, SARS-CoV-2 and MERS) spike protein RBD, suitably a structural mimetic thereof that comprises, consists or consists essentially of the amino acid sequence set forth in SEQ ID NO: 1 or 2, or an amino acid sequence corresponding thereto.

In some embodiments, the ACE2 polypeptide-interacting compound is Evans Blue, which is suitably represented by formula (I):

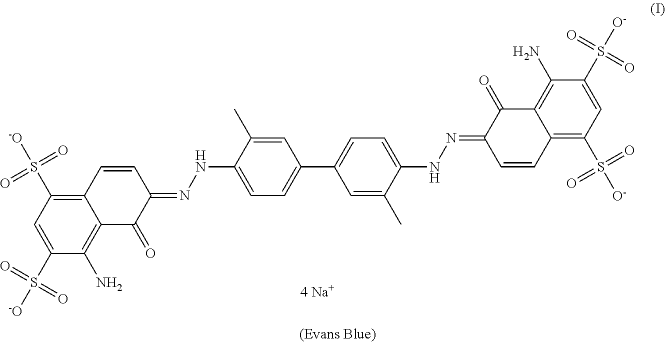

or derivative or prodrug or pharmaceutically acceptable salt thereof.

Derivatives of Evans Blue are known in the art. For example, Chinese patent CN103242255 to Zhang, et al. and International patent application WO2004075925 to Katayama et al., the disclosures of which are incorporated herein by reference in their entirety, disclose truncated derivatives of Evans Blue.

In other embodiments, the ACE2 polypeptide-interacting compound is a benzimidazole derivative as described for example by Guo et al. in U.S. Pat. No. 8,088,368, which is incorporated herein by reference in its entirety. Illustrative derivatives of this type include compounds according to formula (II):

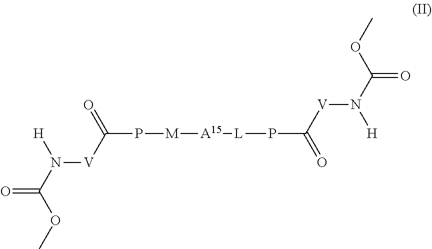

wherein:
V is alkyl; L is benzimidazolyl;
M is a 5-membered heteroaryl ring;
A¹⁵ is:

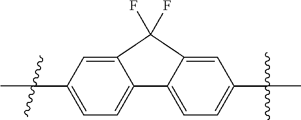 [SMILES: *c1ccc2c(c1)C(F)(F)c1cc(*)ccc1-2]

P is selected from:

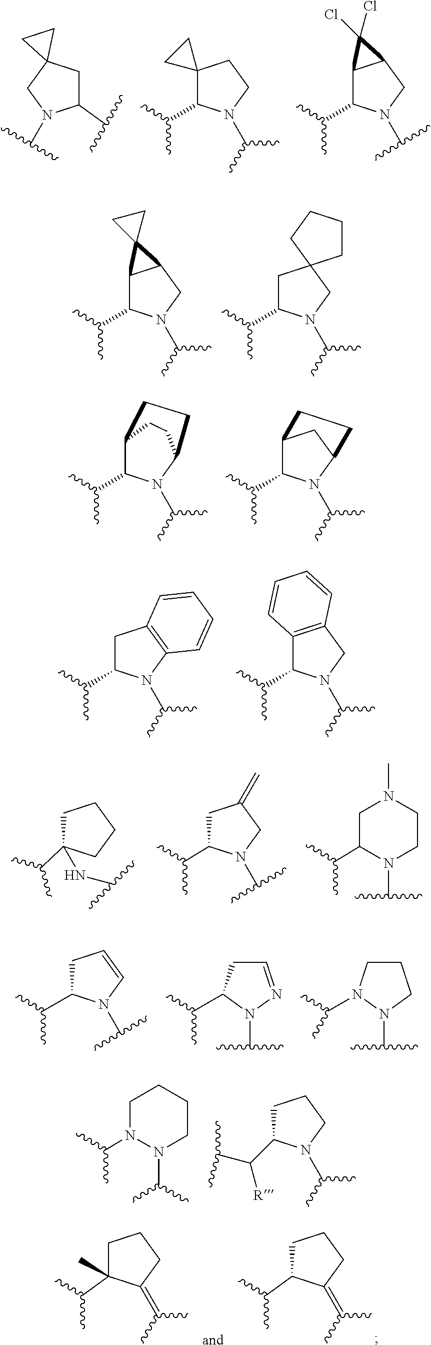

and R′″ is hydrogen or methyl;
or a pharmaceutically acceptable salt, or prodrug thereof.

In specific embodiments, the ACE2 polypeptide-interacting compound is a benzimidazole derivative compound of formula (IIa):

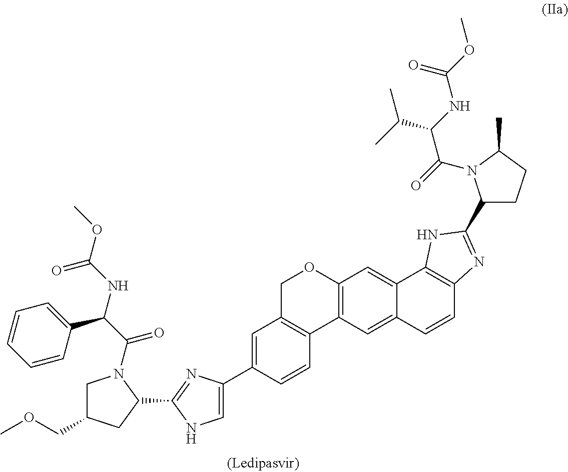

or derivative or prodrug or pharmaceutically acceptable salt thereof.

In some embodiments, the ACE2 polypeptide-interacting compound is a camptothecin derivative as described for example by Miyasaka et al. in European Pat. No. 137145, which is incorporated herein by reference in its entirety. Illustrative derivatives of this type include compounds according to formula (III):

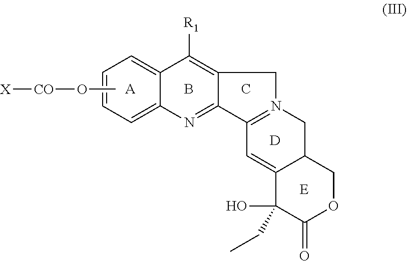

wherein:
R₁ is selected from hydrogen, halogen and alkyl; X is selected from a chlorine atom and NR₂ R₃, wherein R₂ and R₃, which may be identical or different, are selected from a hydrogen atom, optionally substituted alkyl radicals, carbocycles and heterocycles which are optionally substituted, and optionally substituted alkyl radicals, and form, with the nitrogen atom to which they are attached, a heterocycle optionally containing another heteroatom selected from O, S and/or NR₄, R₄ being selected from a hydrogen atom and alkyl radicals; and
wherein the group X—CO—O— is located in position 9, 10 or 11 on ring A,
or a pharmaceutically acceptable salt, or prodrug thereof.

In specific embodiments, the ACE2 polypeptide-interacting compound is a camptothecin derivative of formula (IIIa), wherein X—CO—O— is [4-(1-piperidino)-1-piperidino]carbonyloxy:

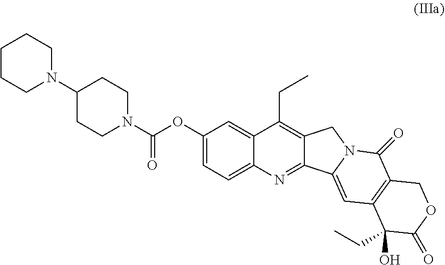 [SMILES: CCc1c2c(nc3ccc(OC(=O)N4CCC(N5CCCCC5)CC4)cc13)-c1cc3c(c(=O)n1C2)COC(=O)[C@]3(O)CC]

or derivative or prodrug or pharmaceutically acceptable salt thereof.

Irinotecan is a prodrug that is metabolized in vivo through cleavage of a carbamate ester bond to form the active drug, SN-38, which has potent topoisomerase I inhibitor activity. In accordance with the present disclosure, it is proposed that SN38 may not have the same ACE2 binding activity as Irinotecan and thus, to improve activity, disclosed herein are compounds based on Irinotecan in which the carbamate ester bond is replaced by an alternate bond that is less labile in vivo.

Thus, also contemplated herein is an ACE2 polypeptide-interacting compound that is a camptothecin derivative of formula (IIIb):

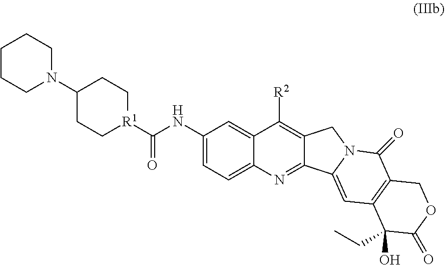

wherein:
R¹ is selected from carbon and heteroatoms, preferably nitrogen; and
R² is selected from hydrogen and C₁₋₆ alkyl, preferably C₁₋₃ alkyl,
or a derivative or pharmaceutically acceptable salt thereof.

In representative examples of this type, the ACE2 polypeptide-interacting compounds are selected from:

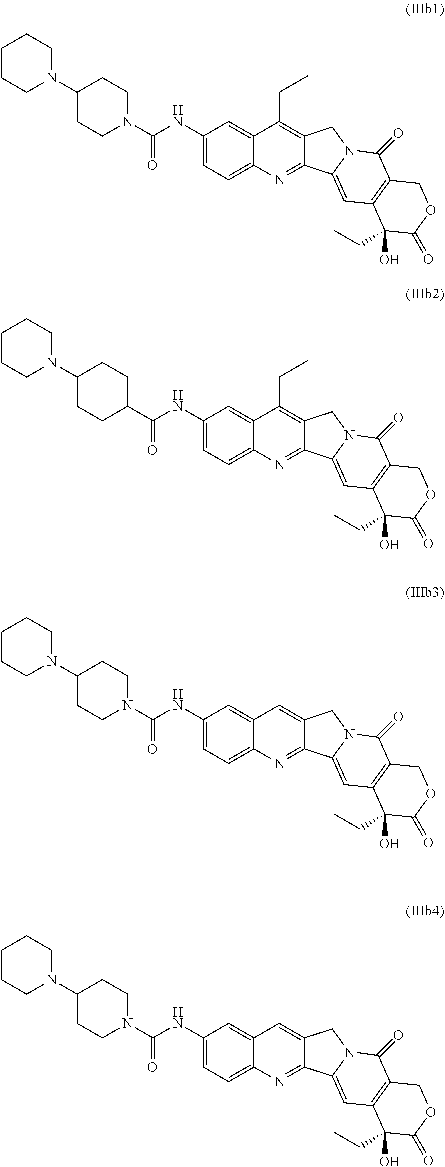 [SMILES: CCc1c2c(nc3ccc(NC(=O)N4CCC(N5CCCCC5)CC4)cc13)-c1cc3c(c(=O)n1C2)COC(=O)[C@]3(O)CC ; CCc1c2c(nc3ccc(NC(=O)C4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2cc3cc(NC(=O)N4CCC(N5CCCCC5)CC4)ccc3nc2-1 ; CC[C@@]1(O)C(=O)OCc2c1cc1n(c2=O)Cc2cc3cc(NC(=O)N4CCC(N5CCCCC5)CC4)ccc3nc2-1]

or a derivative or pharmaceutically acceptable salt thereof.

These camptothecin derivative compounds may be prepared by any suitable technique. In illustrative examples, these compounds are prepared according to the following synthetic schemes:

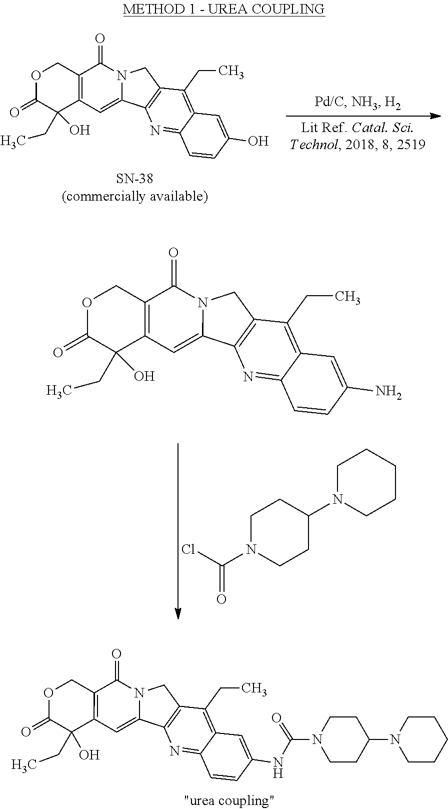 [SMILES: CCc1c2c(nc3ccc(O)cc13)-c1cc3c(c(=O)n1C2)COC(=O)C3(O)CC ; CCc1c2c(nc3ccc(N)cc13)-c1cc3c(c(=O)n1C2)COC(=O)C3(O)CC ; O=C(Cl)N1CCC(N2CCCCC2)CC1 ; CCc1c2c(nc3ccc(NC(=O)N4CCC(N5CCCCC5)CC4)cc13)-c1cc3c(c(=O)n1C2)COC(=O)C3(O)CC]

Commercially available SN-38 is exposed to the conditions described by Cuypers, et al. (Catal. Sci. Technol. 2018, 8, 2519-2523) whereby a high-pressure stainless steel reactor vessel is used, and the substrate is treated with a palladium on carbon catalyst (5 mol %) in the presence of a hydrogen atmosphere and ammonia at 200° C. with agitation. The desired amine product is collected by standard purification techniques (liquid-liquid partition and chromatography).

Coupling between the amine and the acid chloride derivative of 4-piperidinopiperidine would furnish the desired “urea coupled” derivative shown above. The reaction shown above is a standard reaction used to routinely make amide derivatives, and would be completed in the presence of base. Alternatively, it is possible to start with the corresponding carboxylic acid derivative of 4-piperidinopiperidine and do a standard DCC coupling reaction.

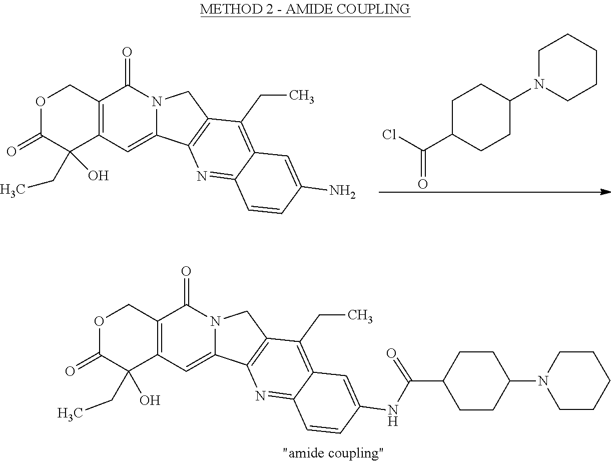 [SMILES: CCc1c2c(nc3ccc(N)cc13)-c1cc3c(c(=O)n1C2)COC(=O)C3(O)CC ; O=C(Cl)C1CCC(N2CCCCC2)CC1 ; CCc1c2c(nc3ccc(NC(=O)C4CCC(N5CCCCC5)CC4)cc13)-c1cc3c(c(=O)n1C2)COC(=O)C3(O)CC]

The amine generated from SN-38 can also be reacted with the cyclohexane substituted analogue of 4-piperidinopiperidine, as shown in the scheme above. The chemistry involved in making this amide coupled analog is the same as for method 1—it is standard amide formation by reaction of an amine with a carboxylic acid or acid chloride, using wither DCC as a coupling agent (for the carboxylic acid) or a base with the acid chloride.

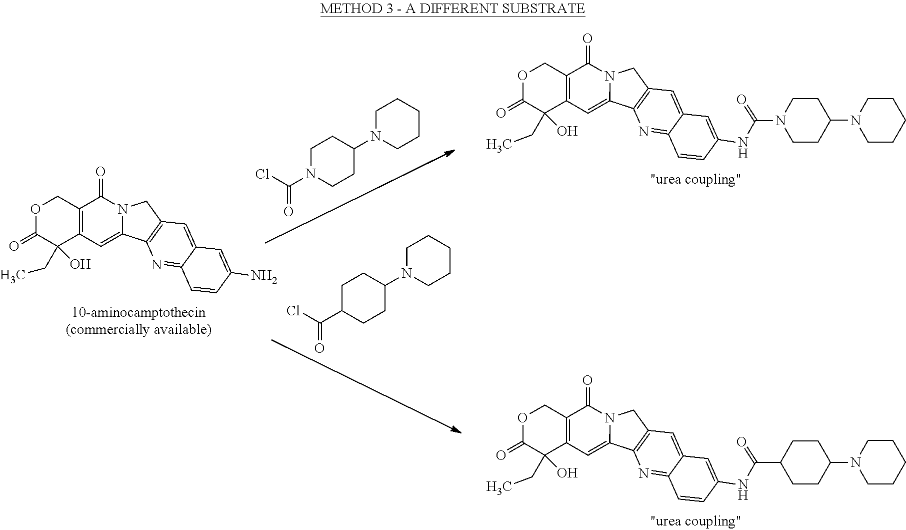 [SMILES: CCC1(O)C(=O)OCc2c1cc1n(c2=O)Cc2cc3cc(N)ccc3nc2-1 ; O=C(Cl)N1CCC(N2CCCCC2)CC1 ; O=C(Cl)C1CCC(N2CCCCC2)CC1 ; CCC1(O)C(=O)OCc2c1cc1n(c2=O)Cc2cc3cc(NC(=O)N4CCC(N5CCCCC5)CC4)ccc3nc2-1 ; CCC1(O)C(=O)OCc2c1cc1n(c2=O)Cc2cc3cc(NC(=O)C4CCC(N5CCCCC5)CC4)ccc3nc2-1]

The same chemistry as above can be utilized to generate a series of analogous 7-desethyl derivatives. 10-Aminocamptothecin is commercially available. Using the identical chemistry protocols to those described for method 1 and method 2 above, coupling of 10-aminocamptothecin with either the acid chloride derivative of 4-piperidinopiperidine or the cyclohexane analogue, would furnish the urea coupled or amide coupled derivatives, respectively.

In other embodiments, the ACE2 polypeptide-interacting compound is a heteropentacyclic compound as described for example by Bacon et al. in U.S. Pat. No. 8,575,135, which is incorporated herein by reference in its entirety. Illustrative compounds of this type include compounds according to formula (IV):

E^{1a}-V^{1a}—C(═O)—P^{1a}—W^{1a}—P^{1b} C(═O)—V^{1b}-E^{1b} (IV)
wherein:
W^{1a} is

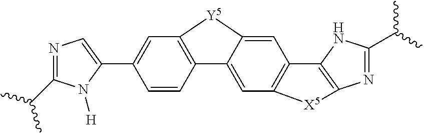

and W^{1a} is optionally substituted with one or more groups independently selected from halo, alkyl, haloalkyl, or cyano;
Y⁵ is —O—CH₂—, or —CH₂—O—; X⁵ is —CH₂—CH₂— or —CH═CH—;
E^{1a} is —N(H)(alkoxycarbonyl), —N(H)(cycloalkylcarbonyl) or —N(H)(cycloalkyloxycarbonyl); or E^{1a}-V^{1a} taken together are R^{9a};
E^{1b} is —N(H)(alkoxycarbonyl), —N(H)(cycloalkylcarbonyl) or —N(H)(cycloalkyloxycarbonyl); or E^{1b}-V^{1b} taken together are R^{9b};
V^{1a} and V^{1b} are each independently selected from:

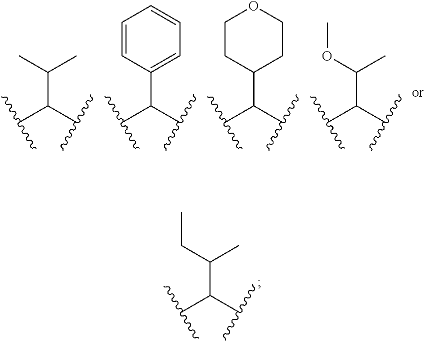

P^{1a} is selected from:

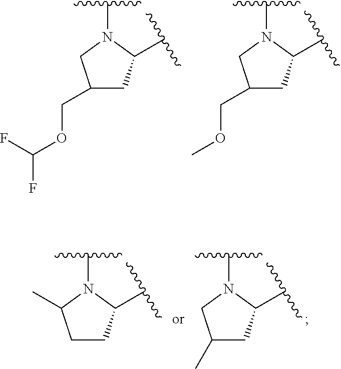

P^{1b} is selected from:

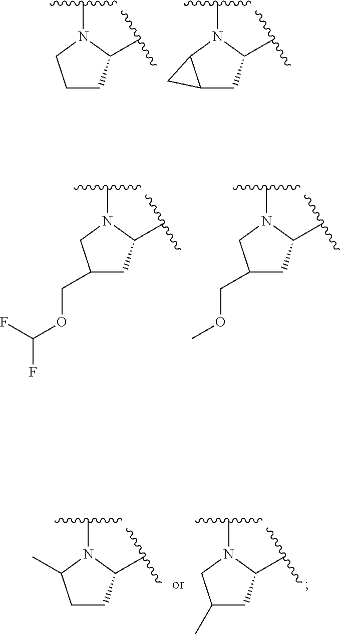

and
R^{9a} and R^{9b} are each independently:

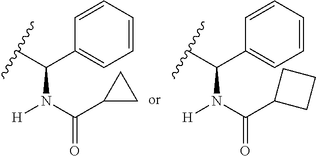 [SMILES: *[C@H](NC(=O)C1CC1)c1ccccc1 ; "or" ; *[C@H](NC(=O)C1CCC1)c1ccccc1]

or a pharmaceutically acceptable salt or prodrug thereof.

In specific embodiments, the ACE2 polypeptide-interacting compound is a heteropentacyclic compound of formula (IVa):

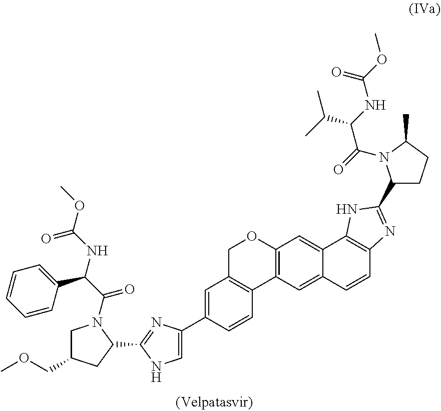

or derivative or prodrug or pharmaceutically acceptable salt thereof.

In still other embodiments, the ACE2 polypeptide-interacting compound is a pyrrolopyridine compound as described for example by Bruncko et al. in U.S. Pat. No. 8,546,399, which is incorporated herein by reference in its entirety. An exemplary compound of this type is a compound of formula (V):

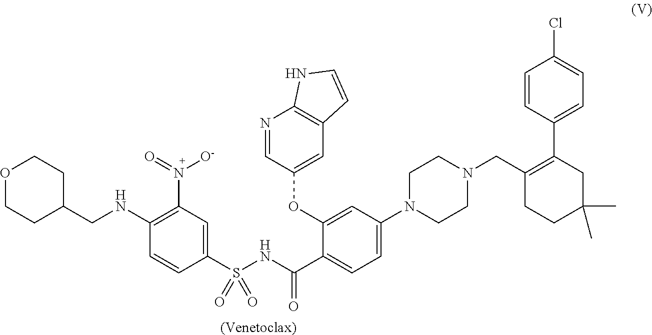

or derivative or prodrug or pharmaceutically acceptable salt thereof.

It will be appreciated that the structures of some of compounds disclosed herein may include asymmetric centers, including asymmetric carbon atoms. It is to be understood accordingly that the isomers arising from such asymmetry (e.g., all enantiomers, stereoisomers, rotamers, tautomers, diastereomers, or racemates) are included within the scope of the disclosure. Such isomers can be obtained in substantially pure form by classical separation techniques or by stereochemically controlled synthesis. Furthermore, the structures and other compounds and moieties discussed in this application also include all tautomers thereof.

Compounds of formulas (I), (II), (III), (IV) and (V) as described herein may be purchased from commercial sources such a chemical manufacturers or suppliers well known to the skilled person. Alternatively, the compounds may be synthesized from commercially available starting materials and/or synthetic intermediates using art recognized synthetic routes. Many of the compounds described are known drug molecules, also referred to as active pharmaceutical ingredients (APIs), and have received regulatory approval for alternative indications to those described herein. Synthetic routes to drug molecules, such as those encompassed by compounds of formulas (I), (II), (III), (IV) and (V), are referenced in the cited patent or scientific publications or are described in, for example, Ruben Vandanyan and Victor Hruby (2006) Synthesis of Essential Drugs (Elsevier Science) or Ruben Vandanyan and Victor Hruby (2016) Synthesis of Best-Seller Drugs (Academic Press), and references therein.

Pharmaceutically acceptable salts are described in, for example, Handbook of Pharmaceutical Salts: Properties, Selection, and Use; Edited by P. Heinrich Stahl and Camile G. Wermuth. VHCA, Verlag Helvetica Chimica Acta, Zürich, Switzerland, and Wiley-VCH, Weinheim, Germany. 2002. Their methods of preparation are well known in the art.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, pamoic acid and the like. For example, an amine group of the compounds of the disclosure may undergo reaction with an acid, for example hydrochloric acid, to form an acid addition salt, for example a hydrochloride or a dihydrochloride.

Pharmaceutically acceptable base addition salts may be prepared from inorganic and organic bases. Corresponding counterions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, choline, betaine, ethylenediamine, glucosamine, N-alkylglucamines, theobromine, purines, piperazine, piperidine, and N-ethylpiperidine. For example, where the compound of the disclosure possesses a carboxylic acid group or a phenol group, the compound may undergo reaction with a base to form the base addition salt. A particular salt is a sodium salt.

In some embodiments, the ACE2 polypeptide-interacting compound is a peptide comprising, consisting or consisting essentially of the amino acid sequence NCYFPLQSYGFQPTNGV [SEQ ID NO: 1] or NCYFPLQSYGFQPTNGVGY [SEQ ID NO: 2], or an amino acid sequence corresponding thereto.

Non-limiting examples of such peptides are represented by the formula (VI):

Z₁—B—Z₂ (VI)
wherein:
Z₁ and Z₂ are independently absent or are independently selected from at least one of a proteinaceous moiety comprising from about 1 to about 50 amino acid residues (and all integer residues in between), and a protecting moiety; and
B is a peptide consisting of the amino acid sequence set forth in SEQ ID NO: 1 or 2, or an amino acid sequence that is distinguished from the amino acid sequence set forth in SEQ ID NO: 1 or 2 by the substitution, deletion or addition of at 1, 2, 3, 4, 5 or 6 amino acids.

In certain embodiments, the peptide of formula (VI) has any one or more activities selected from the group consisting of: (a) inhibits binding between an ACE2-interacting coronavirus and an ACE2 polypeptide-expressing cell; (b) inhibits entry of an ACE2-interacting coronavirus into an ACE2 polypeptide-expressing cell; (c) elicits an immune response to an ACE2-interacting coronavirus in a subject, suitably including production of neutralizing antibodies; (d) detects the presence of an immune response, suitably including a humoral immune response in a subject immunized against an ACE2-interacting coronavirus or exposed to infection with an ACE2-interacting coronavirus; and (e) is able to capture and/or isolate an antigen-binding molecule, suitably including a neutralizing antigen-binding molecule, that binds specifically to an ACE2-interacting coronavirus, preferably the spike protein RBD thereof.

In some embodiments, the peptide of formula (VI) may comprise a primary, secondary or tertiary amide, a hydrazide, a hydroxyamide or a free-carboxyl group at the C-terminus and/or a primary amine or acetamide at the N-terminus. In some embodiments, the peptide of formula (VI) is a cyclic peptide and, thus, may not comprise N- and/or C-terminal amino acid residues.

Peptides of formula (VI) are typically prepared by chemical synthesis. Alternatively, they may be prepared by expression of a coding sequence in suitable host cells, although any suitable methods can be used. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., Escherichia coli, Bacillus subtilis, and Streptococcus spp.), yeast cells (e.g., Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophile) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as Baculovirus systems, are known to those of skill in the art and described in, e.g., Summers and Smith, Texas Agricultural Experiment Station Bulletin No. 1555 (1987). Materials and methods for Baculovirus/insert cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

A peptide coding sequence of the present disclosure is typically expressed using a nucleic acid construct. Nucleic acid constructs are generally prepared in the form of vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable Baculovirus expression vector, such as pFastBac (Invitrogen), can be used to produce recombinant Baculovirus particles. The Baculovirus particles are amplified and used to infect insect cells to express recombinant protein. For expression in mammalian cells, a vector that will drive expression of the construct in the desired mammalian host cell (e.g., Chinese hamster ovary cells) is used.

The peptides can be purified using any suitable method. Suitable methods for purifying desired peptides including precipitation and various types of chromatography, such as hydrophobic interaction, ion exchange, affinity, chelating and size exclusion are well-known in the art. Suitable purification schemes can be created using two or more of these or other suitable methods. If desired, the peptides can include a purification moiety or “tag”, that facilitates purification, as described in Section 5. Such tagged polypeptides can conveniently be purified, for example from conditioned media, by chelating chromatography or affinity chromatography.

### 3. Antigen-Binding Molecules

The peptide compounds disclosed herein are structural mimetics of an ACE2-interacting coronavirus spike protein RBD and in accordance with the present disclosure are useful for producing antigen-binding molecules that bind specifically with an ACE2-interacting coronavirus, suitably the spike protein RBD thereof.

Those of ordinary skill in the art will appreciate the well-developed knowledge base on antigen-binding proteins such as set forth, for example, in Abbas et al., Cellular and Molecular Immunology, 6th ed., W.B. Saunders Company (2010) or Murphey et al., Janeway's Immunobiology, 8th ed., Garland Science (2011), each of which is incorporated herein by reference in its entirety.

In some embodiments, antigen binding proteins that bind specifically with an ACE2-interacting coronavirus are antibodies. Antibodies include intact antibodies and antigen-binding fragments thereof, as described for example in the definition section. An antibody may comprise a complete antibody molecule (including polyclonal, monoclonal, chimeric, humanized, or human versions having full length heavy and/or light chains), or comprise an antigen binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see e.g., Hollinger and Hudson, 2005, Nature Biotechnology, 23, 9, 1126-1136). Also included are antibody polypeptides such as those disclosed in U.S. Pat. No. 6,703,199, including fibronectin polypeptide monobodies. Other antibody polypeptides are disclosed in U.S. Patent Publication 2005/0238646, which are single-chain polypeptides.

Numerous methods of preparing antibodies to antigens of interest are known in the art. For example, monoclonal antibodies that bind specifically with an ACE2-interacting coronavirus can be made using conventional hybridoma methods that are often based on the seminal method of Kohler, G. et al. (1975, “Continuous Cultures Of Fused Cells Secreting Antibody Of Predefined Specificity,” Nature 256:495-497) or a modification thereof. Typically, monoclonal antibodies are developed in non-human species, such as mice. In general, a mouse or rat is used for immunization but other animals may also be used. The antibodies may be produced by immunizing mice with an immunogenic amount of an immunogen, in this case a peptide of the present disclosure, which is a structural mimetic of an ACE2-interacting coronavirus spike protein RBD. The immunogen may be administered multiple times at periodic intervals such as, bi weekly, or weekly, or may be administered in such a way as to maintain viability in the animal.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies that bind specifically to the immunogen, using, for example, FACS (fluorescence activated cell sorting) or immunohistochemistry (IHC) screening. The selected monoclonal antibody-secreting hybridomas are then cultured either in vitro (e.g., in tissue culture bottles or hollow fiber reactors), or in vivo (e.g., as ascites in mice).

As another alternative to the cell fusion technique, Epstein-Barr Virus (EBV)-immortalized B cells may be used to produce monoclonal antibodies that bind specifically with an ACE2-interacting coronavirus. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

Thus, the present disclosure further contemplates methods of producing an antigen-binding molecule that binds specifically with an ACE2-interacting coronavirus, wherein the method comprises: (1) immunizing an animal with a peptide that comprises, consists or consists essentially of the amino acid sequence set forth in SEQ ID NO: 1 or 2, or an amino acid sequence corresponding thereto; (2) detecting a B cell from the animal, which binds specifically with the coronavirus spike protein; and (3) isolating the antigen-binding molecule expressed by that B cell.

The present disclosure also encompasses antigen-binding molecule that are produced by such methods as well as derivatives thereof. Also encompassed are cells including hybridomas that are capable of producing the antigen-binding molecules of the disclosure, and methods of producing antigen-binding molecules from those cells. In specific embodiments, the antigen-binding molecules produced by the methods and cells of the disclosure are preferably neutralizing antigen-binding molecules.

Also contemplated herein are chimeric antibodies and humanized antibodies. In some embodiments, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology 42(12):1445-1451, 2005; Hwang W. et al., Methods 36(1):35-42, 2005; Dall'Acqua W F, et al., Methods 36(1):43-60, 2005; and Clark, M., Immunology Today 21(8):397-402, 2000.

An antibody of the present disclosure may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a subject chimeric polypeptide or complex immunogen.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ:191-200 (2003), Kellermann et al., Curr Opin Biotechnol. 2002, 13:593-97, Russel et al., Infect Immun. 2000, 68:1820-26, Gallo et al., Eur J. Immun. 2000, 30:534-40, Davis et al., Cancer Metastasis Rev. 1999, 18:421-25, Green, J Immunol Methods 1999, 231:11-23, Jakobovits, Advanced Drug Delivery Reviews 1998, 31:33-42, Green et al., J Exp Med. 1998, 188:483-95, Jakobovits A, Exp. Opin. Invest. Drugs 1998, 7:607-14, Tsuda et al., Genomics 1997, 42:413-21, Mendez et al., Nat. Genet. 1997, 15:146-56, Jakobovits, Curr Biol. 1994, 4:761-63, Arbones et al., Immunity 1994, 1:247-60, Green et al., Nat. Genet. 1994, 7:13-21, Jakobovits et al., Nature 1993, 362:255-58, Jakobovits et al., Proc Natl Acad Sci USA 1993, 90:2551-55. Chen, J., M. et al. Int. Immunol. 1993, 5: 647-656, Choi et al., Nature Genetics 1993, 4: 117-23, Fishwild et al., Nature Biotech. 1996, 14: 845-51, Harding et al., 1995, Annals of the New York Academy of Sciences, Lonberg et al., 1994,Nature 368: 856-59, Lonberg, 1994,Transgenic Approaches to Human Monoclonal Antibodies in Handbook of Experimental Pharmacology 113: 49-101, Lonberg et al., Int. Rev. Immunol. 1995, 13: 65-93, Neuberger, Nature Biotech. 1996, 14: 826, Taylor et al., Nucleic Acids Research 1992, 20: 6287-95, Taylor et al., Int. Immunol. 1994, 6: 579-91, Tomizuka et al., Nature Genetics 1997, 16: 133-43, Tomizuka et al., Proc Natl Acad Sci USA 2000, 97: 722-27, Tuaillon et al., Proc Natl Acad Sci USA 1993, 90: 3720-24, and Tuaillon et al., J. Immunol. 1994, 152: 2912-20.; Lonberg et al., Nature 1994, 368:856; Taylor et al., Int. Immunol. 1994, 6:579; U.S. Pat. No. 5,877,397; Bruggemann et al., Curr. Opin. Biotechnol. 1997 8:455-58; Jakobovits et al., Ann. N.Y. Acad. Sci. 1995. 764:525-35. In addition, protocols involving the XenoMouse®. (Abgenix, now Amgen, Inc.) are described, for example in U.S. Ser. No. 05/011,8643 and WO 05/694879, WO 98/24838, WO 00/76310, and U.S. Pat. No. 7,064,244.

Alternatively, the ACE2 polypeptide-interacting peptide compounds disclosed herein may be used to screen for antigen-binding molecules from antigen-binding molecule libraries. For example, an ACE2 polypeptide-interacting peptide compound of the present disclosure may be immobilized to a solid support (e.g., a silica gel, a resin, a derivatized plastic film, a glass bead, cotton, a plastic bead, a polystyrene bead, an alumina gel, or a polysaccharide, a magnetic bead), and screened for binding to antigen-binding molecules. As an alternative, the antigen-binding molecules may be immobilized to a solid support and screened for binding to the ACE2 polypeptide-interacting peptide compound. Any screening assay, such as a panning assay, ELISA, surface plasmon resonance, or other antigen-binding molecule screening assay known in the art may be used to screen for antigen-binding molecules that bind to an ACE2 polypeptide-interacting peptide disclosed herein. The antigen-binding molecule library screened may be a commercially available library, an in vitro generated library, or a library obtained by identifying and cloning or isolating antibodies from an individual infected with an ACE2-interacting coronavirus. In particular embodiments, the antigen-binding molecule library is generated from a survivor of an ACE2-interacting coronavirus outbreak. Antigen-binding molecule libraries may be generated in accordance with methods known in the art. In a particular embodiment, the library is generated by cloning the antibodies and using them in phage display libraries or a phagemid display library.

The present disclosure further encompasses antigen-binding fragments of an anti-ACE2-interactive coronavirus antibody. Such fragments can consist entirely of antibody-derived sequences or can comprise additional sequences. Examples of antigen-binding fragments include Fab, F(ab′)₂, single chain antibodies, diabodies, triabodies, tetrabodies, and domain antibodies. Other examples are provided in Lunde et al., Biochem. Soc. Trans. 2002, 30:500-06.

Single chain antibodies or antigen-binding molecules may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., Prot. Eng. 1997, 10:423; Kortt et al., Biomol. Eng. 2001, 18:95-108). By combining different VL and VH-comprising polypeptides, one can form multimeric scFvs that bind to different epitopes (Kriangkum et al., Biomol. Eng. 2001, 18:31-40). Techniques developed for the production of single chain antibodies include those described in U.S. Pat. No. 4,946,778; Bird, Science 1988, 242:423; Huston et al., Proc. Natl. Acad. Sci. USA 1988, 85:5879; Ward et al., Nature 1989, 334:544, de Graaf et al., Methods Mol. Biol. 2002, 178:379-87.

Antigen-binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 1960, 89:230; Porter, Biochem. J. 1959, 73:119; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another form of an antibody fragment is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a VL and VH domain, which is capable of independently binding a subject ectodomain polypeptide or complex with an affinity at least equal to 10⁻⁷ M or less.

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

Antigen-binding molecules identified in the methods described herein may be tested for neutralizing activity and lack of autoreactivity using biological assays known in the art or described herein. In some embodiments, an antibody isolated from a non-human animal or an antigen-binding molecule library neutralizes a spike protein from more than one ACE2-interacting coronavirus or ACE2-interactive coronavirus strain. In some embodiments, an antigen-binding molecule elicited or identified using an ACE2 polypeptide-interacting peptide compound disclosed herein, neutralizes an ACE2-interactive coronavirus selected from SARS-CoV, SARS-CoV-2 and MERS.

In some embodiments, antigen-binding molecules, including antibodies, elicited or identified using an ACE2 polypeptide-interacting peptide disclosed herein may be used to monitor the efficacy of a therapy and/or disease progression.

Antigen-binding molecules, including antibodies, elicited or identified using an ACE2 polypeptide-interacting peptide may be used in diagnostic immunoassays to detect the presence of an ACE2-interacting coronavirus in biological samples, passive immunotherapy, and generation of antiidiotypic antigen-binding molecules. In addition, the ability of the antigen-binding molecules to neutralize ACE2-interacting coronavirus spike protein and the specificity of the antigen-binding molecules for the spike protein may be tested prior to using the antibodies in passive immunotherapy.

Specific binding of an antigen-binding molecule to an ACE2 polypeptide-interacting peptide disclosed herein and cross-reactivity with other antigens can be assessed by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York).

In some embodiments, the antigen-binding molecules disclosed herein are used in immunodetection methods for binding, purifying, removing, quantifying and otherwise generally detecting ACE2-interacting coronavirus. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The immunodetection methods also include methods for detecting and quantifying the amount of ACE2-interacting coronavirus or related components (e.g., spike protein thereof) in a sample and the detection and quantification of any immune complexes formed during the binding process. In non-limiting examples, a sample suspected of containing ACE2-interacting coronavirus is obtained from a patient, and the sample is contacted with an antigen-binding molecule that binds specifically to the spike RBD of an ACE2-interacting coronavirus, followed by detecting and quantifying the amount of immune complexes formed under the specific conditions. In terms of antigen detection, the biological sample analyzed may be any sample that is suspected of containing an ACE2-interacting coronavirus, such as a tissue section or specimen, a homogenized tissue extract, a biological fluid, including a biological fluid and/or tissue obtained or derived from the respiratory tract including mouth, nose, throat and lungs.

Contacting the chosen biological sample with the antigen-binding molecule under suitable conditions and for a period of time sufficient to allow the formation of immune complexes (primary immune complexes) is generally a matter of simply adding the antigen-binding molecule to the sample and incubating the mixture for a period of time long enough for the antigen-binding molecule to form immune complexes with, i.e., to bind to ACE2-interacting coronavirus or related components (e.g., spike protein thereof) present in the sample. After this time, the sample-antigen-binding molecule composition, such as a tissue section, ELISA plate, dot blot or Western blot, will generally be washed to remove any non-specifically bound antigen-binding molecule species, allowing only those antigen-binding molecule specifically bound within the primary immune complexes to be detected.

### 4. Pharmaceutical Compositions

While it is possible that, for use in therapy, an ACE2 polypeptide-interacting compound described herein may be administered in an undiluted form, it is preferable to present such a compound as a pharmaceutical composition.

A pharmaceutical composition may comprise an ACE2 polypeptide-interacting compound described herein and a pharmaceutically acceptable carrier. Carriers must be “acceptable” in the sense of being compatible with the other ingredients of the composition and not deleterious to the recipient thereof.

In accordance with the disclosure, a compound as described is administered under a therapeutic regime that is non-toxic to the subject.

The pharmaceutical compositions of the present disclosure or the compositions used in the methods of the present disclosure may be formulated and administered using methods known in the art. Techniques for formulation and administration may be found in, for example, Remington: The Science and Practice of Pharmacy, Loyd V. Allen, Jr (Ed), The Pharmaceutical Press, London, 22^{nd} Edition, September 2012.

The compositions of the disclosure may be formulated for administration by any route. In some embodiments the composition is formulated for oral administration. An oral composition may be in the form of tablets, capsules, powders, granules, or liquid preparations. In some embodiments the composition is formulated for topical administration. A topical composition may be in the form of a cream, lotion, ointment, or gel. In some embodiments the composition is formulated for parenteral administration, for example by an intramuscular, intrathecal, intraperitoneal, intravaginal, intrauterine, intravesical or intravenous route.

Suitable unit dosages and maximum daily dosages of an ACE2 polypeptide-interacting compound described herein may be determined in accordance with the unit doses and maximum daily doses used conventionally. Accordingly, the ACE2 polypeptide-interacting compound described herein may be administered to a patient at a daily dosage of, for example, from 250 mg to 750 mg every 6 hours to 500 mg to 1 g every 6 to 8 hours, with a maximum dose of approximately 50 mg/Kg/day or 4 g/day.

An ACE2 polypeptide-interacting compound as described herein, may be the sole active ingredient administered to the subject. However, it will be appreciated that the compound may be administered with an ancillary agent (e.g., a further therapeutic agent such as an antimicrobial agent). Accordingly, the present disclosure contemplates administering an ACE2 polypeptide-interacting compound described herein with one or more further ancillary agents in combination. The combination may allow for concurrent administration (e.g., separate, sequential or simultaneous administration) of the compound with the other active ingredient(s). The combination may be provided in the form of a pharmaceutical composition. Administration with one or more other active ingredients is within the scope of the disclosure. In specific embodiments, an ACE2 polypeptide-interacting compound described herein may be administered concurrently with an antimicrobial agent, which includes without limitation compounds that kill or inhibit the growth of microorganisms such as viruses, bacteria, yeast, fungi, protozoa, etc. and thus include antibiotics, antifungals, antiprotozoals, antimalarials, antituberculotics and antivirals. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin; gemifloxacin; and garenoxacin), tetracyclines, glycylcyclines and oxazolidinones (e.g., chlortetracycline, demeclocycline, doxycycline, lymecycline, methacycline, minocycline, oxytetracycline, tetracycline, tigecycline; linezolide, eperozolid), glycopeptides, aminoglycosides (e.g., amikacin, arbekacin, butirosin, dibekacin, fortimicins, gentamicin, kanamycin, meomycin, netilmicin, ribostamycin, sisomicin, spectinomycin, streptomycin, tobramycin), β-lactams (e.g., imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefinetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol. Representative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, valganciclovir, zalcitabine, zanamivir, and zidovudine. Non-limiting examples antiprotozoals include atovaquone, chloroquine hydrochloride, chloroquine phosphate, metronidazole, metronidazole hydrochloride, and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, bifonazole, butoconazole, chlordantoin, chlorphenesin, ciclopirox olamine, clotrimazole, eberconazole, econazole, fluconazole, flucytosine, flutrimazole, griseofulvin microsize, griseofulvin ultramicrosize, itraconazole, isoconazole, itraconazole, ketoconazole, miconazole, nifuroxime, nystatin, terbinafine hydrochloride, tioconazole, terconazole and undecenoic acid. Non-limiting examples of antimalarials include chloroquine hydrochloride, chloroquine phosphate, doxycycline, hydroxychloroquine sulfate, mefloquine hydrochloride, primaquine phosphate, pyrimethamine, and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the compound care should be taken to ensure that the activity of the compound is not destroyed in the process and that the compound is able to reach its site of action without being destroyed. In some circumstances it may be necessary to protect the compound by means known in the art, such as, for example, micro encapsulation or coating (such as the use of enteric coating). Similarly the route of administration chosen should be such that the compound reaches its site of action.

Those skilled in the art may readily determine appropriate formulations for the compounds of the present disclosure using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, and reducing agents such as methionine or sulfite.

The ACE2 polypeptide-interacting compounds described herein, or pharmaceutically acceptable salts thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminum monostearate and gelatin. Pharmaceutical forms suitable for injectable use may be delivered by any appropriate route including intravenous, intramuscular, intracerebral, intrathecal, epidural injection, intravesicular administration or infusion.

Sterile injectable solutions are prepared by incorporating the active compound in the required amount in the appropriate solvent with various of the other ingredients such as those enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of an ACE2 polypeptide-interacting compound described herein, in which the active compound may be formulated with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, and a sweetening agent, preservative, dye or flavoring.

Any component used in the preparation of any dosage unit form should be pharmaceutically pure and substantially non-toxic in the amounts employed.

The present disclosure also extends to any other forms suitable for administration, for example topical application such as creams, foams, washes, lotions, sprays, and gels; enteral formulations such as suppositories; or compositions suitable for inhalation or intranasal delivery, for example solutions, aerosols, dry powders, suspensions or emulsions. In specific embodiments, the compounds of the present disclosure are formulated for topical application to the skin or body cavity, such as foams, creams, washes, gels, sprays, suppositories, pessaries, lotions, ointment, ovule, tampon, or aerosol. In specific embodiments, an ACE2 polypeptide-interacting compound described herein is formulated for delivery to the respiratory tract and may be administered for example intranasally, inhalationally, intratracheally, intrapulmonarylly or intrabronchially. In representative examples of this type, an ACE2 polypeptide-interacting compound described herein may be administered for example using an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It may be advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the disclosure are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 200 mg. Expressed in proportions, the active compound may be present in from about 0.25 μg to about 200 mg/mL of carrier. In the case of compositions containing supplementary active ingredients, the dosages are determined by reference to the usual dose and manner of administration of the said ingredients.

In some embodiments relating to the ACE2 polypeptide-interacting peptide compounds disclosed herein, the compounds are used as immune-modulating agents for eliciting an immune response to an ACE2-interacting coronavirus. Such “immune-modulating” or “immunogenic” compositions include preventative compositions (i.e., compositions administered for the purpose of preventing a condition such as an infection) and therapeutic compositions (i.e., compositions administered for the purpose of treating conditions such as an infection). An immunomodulating composition of the present disclosure may therefore be administered to a recipient for prophylactic, ameliorative, palliative, or therapeutic purposes.

Supplementary active ingredients such as adjuvants or biological response modifiers can also be incorporated into immunogenic compositions of the present disclosure. Although adjuvant(s) may be included in pharmaceutical compositions of the present disclosure they need not necessarily comprise an adjuvant. In such cases, reactogenicity problems arising from the use of adjuvants may be avoided.

In general, adjuvant activity in the context of a pharmaceutical composition of the present disclosure includes, but is not limited to, an ability to enhance the immune response (quantitatively or qualitatively) induced by immunogenic components in the composition (e.g., an ACE2 polypeptide-interacting peptide compound disclosed herein). This may reduce the dose or level of the immunogenic components required to produce an immune response and/or reduce the number or the frequency of immunizations required to produce the desired immune response.

Any suitable adjuvant may be included in a pharmaceutical composition of the present disclosure. For example, an aluminum-based adjuvant may be utilized. Suitable aluminum-based adjuvants include, but are not limited to, aluminum hydroxide, aluminum phosphate and combinations thereof. Other specific examples of aluminum-based adjuvants that may be utilized are described in European Pat. No. 1216053 and U.S. Pat. No. 6,372,223. Other suitable adjuvants include Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; oil in water emulsions including those described in European Patent No. 0399843, U.S. Pat. No. 7,029,678 and PCT Publication No. WO 2007/006939; and/or additional cytokines, such as GM-CSF or interleukin-2, −7, or −12, granulocyte-macrophage colony-stimulating factor (GM-CSF), tumor necrosis factor (TNF) monophosphoryl lipid A (MPL), cholera toxin (CT) or its constituent subunit, heat labile enterotoxin (LT) or its constituent subunit, toll-like receptor ligand adjuvants such as lipopolysaccharide (LPS) and derivatives thereof (e.g., monophosphoryl lipid A and 3-Deacylated monophosphoryl lipid A), Flavivirus NS1 and muramyl dipeptide (MDP).

Pharmaceutical compositions of the present disclosure may be provided in a kit. The kit may comprise additional components to assist in performing the methods of the present disclosure such as, for example, administration device(s), buffer(s), and/or diluent(s). The kits may include containers for housing the various components and instructions for using the kit components in the methods of the present disclosure.

### 5. Platforms, Assays and Device for Using the ACE2 Polypeptide-Interacting Peptides

The present disclosure encompasses ACE2 polypeptide-interacting peptide compounds that comprise, consist or consist essentially of the amino acid sequence set forth in SEQ ID NO: 1 or 2, or an amino acid sequence corresponding thereto. These compounds are disclosed herein to mimic structural determinants of an ACE2-interacting coronavirus spike protein RBD, which are predicted to be reactive with, and sensitive to, antibodies to ACE2-interacting coronaviruses in patient samples. These peptides can be used in any type of serological assay or platform, now known or later developed, to screen for the presence of antibodies to ACE2-interacting coronaviruses and to determine if a subject has had an infection by and/or exposure to an ACE2-interacting coronavirus. These peptides can also be used to test for and monitor humoral responses to immune-modulating compositions, including vaccines, and immunomodulatory drugs, thus, being useful for the development of treatment and preventative agents for ACE2-interacting coronaviruses.

There are a number of different conventional assays for detecting formation of an antibody-peptide complex comprising one or more peptide compounds disclosed herein. For example, the detecting step can comprise performing an ELISA assay, performing an immunofluorescence assay, performing a lateral flow immunoassay, performing an agglutination assay, performing a wavelength shift assay, performing a Western blot, slot blot, or dot blot, analyzing the sample in an analytical or centrifugal rotor, or analyzing the sample with an electrochemical, optical, or opto-electronic sensor. These different assays are described herein and/or are well-known to those skilled in the art.

Thus, the ACE2 polypeptide-interacting peptides of the present disclosure can be used in any assay, format or platform for antibody detection including but not limited to ELISA, Luminex, Western blot assays, and spotted peptide arrays, as well as those platforms that are later developed.

In certain embodiments of the disclosure, the assay comprises immobilizing the antibody(s) in the sample; adding a peptide of the disclosure; and detecting the degree of antibody bound to the peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

In other embodiments, the assay comprises: immobilizing a peptide of the disclosure; adding the sample containing antibodies; and detecting the amount of antibody bound to the peptide, e.g., by adding another peptide of the disclosure conjugated, directly or indirectly, to a label (e.g., metallic nanoparticle or metallic nanoshell, fluorescent label, or enzyme (e.g., horseradish peroxidase or alkaline phosphatase)) or by adding a labeled substance, such as a binding partner or a labeled antibody which specifically recognizes the sample antibodies (e.g., anti-human IgG antibodies, or anti-human IgM antibodies).

In other embodiments, the assay comprises: immobilizing a peptide of the disclosure; adding the sample containing antibodies; and detecting the amount of antibody bound to the peptide, e.g., by adding a first binding partner which specifically recognizes the sample antibodies (e.g., anti-human IgG antibodies, or anti-human IgM antibodies), and further adding a second binding partner, wherein the second binding partner is labeled and recognizes said first binding partner.

In still other embodiments, the assay comprises: reacting the peptide and the sample containing antibodies without any of the reactants being immobilized, and then detecting the amount of complexes of antibody and peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

Immobilization of a peptide of the disclosure can be either covalent or non-covalent, and the non-covalent immobilization can be non-specific (e.g., non-specific binding to a polystyrene surface in a microtiter well). Specific or semi-specific binding to a solid or semi-solid carrier, support or surface, can be achieved by the peptide having, associated with it, a moiety which enables its covalent or non-covalent binding to the solid or semi-solid carrier, support or surface. For example, the moiety can have affinity to a component attached to the carrier, support or surface. In this case, the moiety may be, for example, a biotin or biotinyl group or an analog thereof bound to an amino acid group of the peptide, and the component is then avidin, streptavidin, neutravidin, or an analogue thereof.

Suitable carriers, supports, and surfaces include, but are not limited to, metallic nanolayers, beads (e.g., magnetic beads, colloidal particles or metallic nanoparticles or nanoshells, such as colloidal gold, or particles or nanoparticles comprising silica, latex, polystyrene, polycarbonate, or PDVF), latex of co-polymers such as styrene-divinyl benzene, hydroxylated styrene-divinyl benzene, polystyrene, carboxylated polystyrene, beads of carbon black, non-activated or polystyrene or polyvinyl chloride activated glass, epoxy-activated porous magnetic glass, gelatin or polysaccharide particles or other protein particles, red blood cells, mono- or polyclonal antibodies or Fab fragments of such antibodies.

The protocols for immunoassays using antigens for detection of specific antibodies are well known in art. For example, a conventional sandwich assay can be used, or a conventional competitive assay format can be used.

Devices for performing specific binding assays, especially immunoassays, are known and can be readily adapted for use in the present methods. Solid-phase assay devices include microtiter plates, flow-through assay devices (e.g., lateral flow immunoassay devices), dipsticks, and immunocapillary or immunochromatographic immunoassay devices.

In embodiments of the disclosure, the solid or semi-solid surface or carrier is the floor or wall in a microtiter well, a filter surface or membrane (e.g., a nitrocellulose membrane or a PVDF (polyvinylidene fluoride) membrane), a hollow fiber, a beaded chromatographic medium (e.g., an agarose or polyacrylamide gel), a magnetic bead, a fibrous cellulose matrix, an HPLC matrix, an FPLC matrix, a substance having molecules of such a size that the molecules with the peptide bound thereto, when dissolved or dispersed in a liquid phase, can be retained by means of a filter, a substance capable of forming micelles or participating in the formation of micelles allowing a liquid phase to be changed or exchanged without entraining the micelles, a water-soluble polymer, or any other suitable carrier, support or surface.

In some embodiments disclosed herein, the peptide of the disclosure is provided with a suitable label which enables detection. Conventional labels may be used which are capable, alone or in concert with other compositions or compounds, of providing a detectable signal.

Suitable labels include, but are not limited to, enzymes (e.g., HRP, beta-galactosidase, or alkaline phosphatase), fluorescent labels, radioactive labels, colored latex particles, and metal-conjugated labels (e.g., metallic nanolayers, metallic nanoparticle- or metallic nanoshell-conjugated labels). Suitable metallic nanoparticle or metallic nanoshell labels include, but are not limited to, gold particles, silver particles, copper particles, platinum particles, cadmium particles, composite particles, gold hollow spheres, gold-coated silica nanoshells, and silica-coated gold shells. Metallic nanolayers suitable for detectable layers include nanolayers comprised of cadmium, zinc, mercury, and noble metals, such as gold, silver, copper, and platinum.

Suitable detection methods include, but are not limited to, detection of an agent which is tagged, directly or indirectly, with a colorimetric assay (e.g., for detection of HRP or beta-galactosidase activity), visual inspection using light microscopy, immunofluorescence microscopy, including confocal microscopy, or by flow cytometry (FACS), autoradiography (e.g., for detection of a radioactively labeled agent), electron microscopy, immunostaining, subcellular fractionation, or the like. In one embodiment, a radioactive element (e.g., a radioactive amino acid) is incorporated directly into a peptide chain. In another embodiment, a fluorescent label is associated with a peptide via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L or combinations thereof) which binds to the first antibody. This secondary antibody or other binding agent can be labeled with, for example, a radioactive, enzymatic, fluorescent, luminescent, metallic nanoparticle or metallic nanoshell (e.g. colloidal gold), or other detectable label, such as an avidin/biotin system. In another embodiment, the binding partner is a peptide of the disclosure, which can be conjugated directly or indirectly to an enzyme, such as horseradish peroxidase or alkaline phosphatase or other signaling moiety. In such embodiments, the detectable signal is produced by adding a substrate of the enzyme that produces a detectable signal, such as a chromogenic, fluorogenic, or chemiluminescent substrate.

In some embodiments of the disclosure, the detection procedure comprises visibly inspecting the antibody-peptide complex for a color change, or inspecting the antibody-peptide complex for a physical-chemical change. Physical-chemical changes may occur with oxidation reactions or other chemical reactions. They may be detected by eye, using a spectrophotometer, or the like.

One assay format is a lateral flow immunoassay format. Antibodies to human or animal immunoglobulins, can be labeled with a signal generator or reporter (e.g., colloidal gold) that is dried and placed on a glass fiber pad (sample application pad or conjugate pad).

A diagnostic peptide as disclosed herein may be immobilized on a membrane, such as nitrocellulose or a PVDF (polyvinylidene fluoride) membrane. When a sample is applied to the sample application pad (or flows through the conjugate pad), it dissolves the labeled reporter, which then binds to all antibodies in the sample. The resulting complexes are then transported into the next membrane (PVDF or nitrocellulose containing the diagnostic peptide) by capillary action. If antibodies against the diagnostic peptide are present, they bind to the diagnostic peptide striped on the membrane, thereby generating a signal (e.g., a band that can be seen or visualized). An additional antibody specific to the labeled antibody or a second labeled antibody can be used to produce a control signal.

An alternative format for the lateral flow immunoassay comprises the peptides or compositions of the disclosure being conjugated to a ligand (e.g., biotin) and complexed with labeled ligand receptor (e.g., streptavidin-colloidal gold). The labeled peptide complexes can be placed on the sample application pad or conjugate pad. Anti-human IgG/IgM or anti-animal IgG/IgM antibodies of the disclosure are immobilized on a membrane, such as nitrocellulose of PVDF, at a test site. When sample is added to the sample application pad, antibodies in the sample react with the labeled peptide complexes such that antibodies that bind to peptides of the disclosure become indirectly labeled. The antibodies in the sample are then transported into the next membrane (PVDF or nitrocellulose containing the diagnostic peptide) by capillary action and bind to the immobilized anti-human IgG/IgM or anti-animal IgG/IgM antibodies. If any of the sample antibodies are bound to the labeled peptides of the disclosure, the label associated with the peptides can be seen or visualized at the test site.

Another assay for the screening of blood products or other physiological or biological fluids is an enzyme linked immunosorbent assay, i.e., an ELISA. Typically in an ELISA, isolated peptides or collection or set of peptides of the disclosure, are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with a biological sample suspected of containing specific antibody. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody that is conjugated to an enzyme or other label by standard procedures and is dissolved in blocking buffer. The label can be chosen from a variety of enzymes, including horseradish peroxidase (HRP), beta-galactosidase, alkaline phosphatase (ALP), and glucose oxidase. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length). Conditions for performing ELISA assays are well-known in the art.

In another embodiment of an ELISA, a peptide or a collection or set of peptides of the disclosure is immobilized on a surface, such as a ninety-six-well ELISA plate A sample is then added and the assay proceeds as above.

In still other embodiments, a peptide or collection or set of peptides of the disclosure are electro- or dot-blotted onto nitrocellulose paper. Subsequently, a sample, such as a biological fluid (e.g., serum or plasma) is incubated with the blotted antigen, and antibody in the biological fluid is allowed to bind to the antigen(s). The bound antibody can then be detected, e.g., by standard immunoenzymatic methods or by visualization using metallic nanoparticles or nanoshells coupled to secondary antibodies or other antibody binding agents or combinations thereof.

It should be understood by one of skill in the art that any number of conventional protein assay formats, particularly immunoassay formats, may be designed to utilize the ACE2-interacting peptide compounds of this disclosure for the detection of ACE2-interacting coronavirus antibodies in a subject. This disclosure is thus not limited by the selection of the particular assay format, and is believed to encompass assay formats that are known to those of skill in the art. To date most serology has been performed using singleplex ELISA, complement fixation or neutralization assays. More recently, Luminex-based systems have been employed that can address up to 100 antigenic targets simultaneously (i.e., 100 individual pathogens, 100 individual antigenic targets for one pathogen, or some variation thereof) (Anderson et al. J. Immunol. Methods 2011, 366:79-88). Additionally, arrays are established that comprise spotted recombinant proteins expressed in vitro in E. coli, S. cerevisiae, baculoviruses, or cell-free, coupled transcription-translation systems (Vigil et al. Future Microbiol. 2010, 5:241-51.).

In some embodiments, the ACE2-interacting peptide compounds of the present disclosure can be used to isolate and/or purify antigen-binding molecules (e.g., antibodies), suitably neutralizing antigen binding molecules, which bind specifically to an ACE2-interactive coronavirus, including to the spike protein RBD thereof. Any suitable technique may be used, including for example immunoaffinity purification using standard techniques and one or more peptide compounds of the present disclosure as an affinity reagent. The affinity resin used can be an activated CH-Sepharose coupled to the one or more of the peptide compounds of the present disclosure. Antigen-binding molecule-containing samples can be loaded onto the column and washed with PBS or another appropriate buffer or washing solution. The antigen-binding molecules can then be eluted and collected. The concentration of antigen-binding molecules obtained can be determined using a total protein colorimetric determination (Bio-Rad).

### 6. Methods and Systems for Serological Detection of Exposure to ACE2-Interacting Coronavirus

The present disclosure includes methods and systems for the detection of exposure to ACE2-interacting coronavirus, i.e., antibodies to an ACE2-interacting coronavirus, including its spike protein RBD, in any sample utilizing the peptides disclosed herein.

Suitable methods typically include: receiving or obtaining (e.g., from a patient) a sample of bodily fluid or tissue likely to contain antibodies; contacting (e.g., incubating or reacting) a sample to be assayed with a peptide or peptides of the disclosure, under conditions effective for the formation of a specific peptide-antibody complex (e.g., for specific binding of the peptide to the antibody); and assaying the contacted (reacted) sample for the presence of an antibody-peptide reaction (e.g., determining the amount of an antibody-peptide complex). The presence of the antibody-peptide complex indicates that the subject was exposed to and infected by an ACE2-interacting coronavirus. A peptide, including a modified form thereof, which “binds specifically” to an antibody against an ACE2-interacting coronavirus spike protein RBD interacts with the antibody, or forms or undergoes a physical association with it, in an amount and for a sufficient time to allow detection of the antibody.

Conditions for reacting peptides and antibodies so that they react specifically are well-known to those of skill in the art. See, e.g., Current Protocols in Immunology (Coligan et al., editors, John Wiley & Sons, Inc).

Once the peptide or peptides of the disclosure and sample antibody are permitted to react in a suitable medium, an assay is performed to determine the presence or absence of an antibody-peptide reaction. Any of the assays discussed herein can be used.

The methods and systems of the present disclosure may be used to detect exposure to ACE2-interacting coronaviruses in research and clinical settings.

One sample for use in the methods is a biological sample. A biological sample may be obtained from a tissue of a subject or bodily fluid from a subject including but not limited to nasopharyngeal aspirate, blood, cerebrospinal fluid, saliva, serum, plasma, urine, sputum, bronchial lavage, pericardial fluid, or peritoneal fluid, or a solid such as feces. Preferred biological samples include biological fluids and/or tissues obtained or derived from the respiratory tract including mouth, nose, throat and lungs, representative examples of which include: sputum; nasopharyngeal aspirate; and bronchial aspirate. The subject may be any animal, particularly a vertebrate and more particularly a mammal, including, without limitation, a cow, dog, human, monkey, mouse, pig, or rat. In one embodiment, the subject is a human. A sample may also be a research, clinical, or environmental sample, such as cells, cell culture, cell culture medium, and compositions for use as, or the development of pharmaceutical and therapeutic agents.

Additional applications include, without limitation, detection of the screening of blood products (e.g., screening blood products for infectious agents), biodefense, food safety, environmental contamination, forensics, and genetic-comparability studies. The present disclosure also provides methods and systems for detecting viral antibodies in cells, cell culture, cell culture medium and other compositions used for the development of pharmaceutical and therapeutic agents.

The subject may have been exposed to an ACE2-interacting coronavirus, suspected of having exposure to an ACE2-interacting coronavirus or believed not to have had exposure to an ACE2-interacting coronavirus. In some embodiments, the subject may have been found to be seropositive by ACE2-interacting coronavirus ELISA.

In certain embodiments, the subject may be a test subject, which has been administered a ACE2-interacting coronavirus vaccine or immunomodulatory agent.

The systems and methods described herein support the detection and measure of a humoral immune response to an ACE2-interacting coronavirus.

### 7. Kits

The present disclosure also contemplates reagents and kits for practicing the methods of the disclosure. These reagents and kits may vary.

One reagent of the kit would be one or more ACE2 polypeptide-interacting peptides of the present disclosure that comprise, consist or consist essentially of the amino acid sequence set forth in SEQ ID NO: 1 or 2, or an amino acid sequence corresponding thereto, which are reactive with, and specific for ACE2-interacting coronavirus antibodies.

In certain embodiments, the peptides are attached to or immobilized on a solid support. In some embodiments, the peptides are attached to or immobilized on a solid support through a metallic nanolayer (e.g., cadmium, zinc, mercury, gold, silver, copper, or platinum nanolayer). In certain embodiments, the solid support is a bead (e.g., a colloidal particle or a metallic nanoparticle or nanoshell), a flow path in a lateral flow immunoassay device, a flow path in an analytical or centrifugal rotor, a tube or a well (e.g., in a plate), or a sensor (e.g., an electrochemical, optical, or opto-electronic sensor).

Reagents for particular types of assays can also be provided in kits of the disclosure. Thus, the kits can include a population of beads (e.g., suitable for an agglutination assay or a lateral flow assay), or a plate (e.g., a plate suitable for an ELISA assay). In other embodiments, the kits comprise a device, such as a lateral flow immunoassay device, an analytical or centrifugal rotor, a Western blot, a dot blot, a slot blot, or an electrochemical, optical, or opto-electronic sensor.

In addition, the kits can include various diluents and buffers, labeled conjugates or other agents for the detection of specifically bound antigens or antibodies (e.g. labeling reagents), and other signal-generating reagents, such as enzyme substrates, cofactors and chromogens. In some embodiments, the kit comprises an anti-human IgG/IgM antibody conjugated to a detectable label (e.g., a metallic nanoparticle, metallic nanoshell, metallic nanolayer, fluorophore, colored latex particle, or enzyme) as a labeling reagent. In other embodiments, the kit comprises protein A, protein G, protein A/G fusion proteins, protein L, or combinations thereof conjugated to a detectable label (e.g., a metallic nanoparticle, metallic nanoshell, metallic nanolayer, fluorophore, colored latex particle, or enzyme) as a labeling reagent. In still other embodiments, the labeling reagents of the kit are a second collection or set of peptides of the disclosure conjugated to a detectable label (e.g., a metallic nanoparticle, metallic nanoshell, metallic nanolayer, fluorophore, colored latex particle, or enzyme). The second collection or set of peptides can be the same as or different than the collection or set of peptides, which may optionally be attached to or immobilized upon a solid support.

Other components of a kit can easily be determined by one of skill in the art. Such components may include coating reagents, polyclonal or monoclonal capture antibodies specific for a peptide of the disclosure, or a cocktail of two or more of the antibodies, purified or semi-purified extracts of these antigens as standards, monoclonal antibody detector antibodies, an anti-mouse, anti-dog, anti-cat, anti-chicken, or anti-human antibody conjugated to a detectable label, indicator charts for colorimetric comparisons, disposable gloves, decontamination instructions, applicator sticks or containers, a sample preparatory cup and buffers or other reagents appropriate for constituting a reaction medium allowing the formation of a peptide-antibody complex.

Such kits provide a convenient, efficient way for a clinical laboratory to diagnose infection by an ACE2-interacting coronavirus. Thus, in certain embodiments, the kits further comprise instructions.

### 8. Screening Assays

The present disclosure further encompasses the use of the peptide compounds disclosed herein, which are structural mimetics of an ACE2-interacting coronavirus spike protein RBD, in screening assays for identifying agents that inhibit interaction of an ACE2-interacting coronavirus with an ACE2 polypeptide-expressing cell, or that inhibit entry of an ACE2-interacting coronavirus into an ACE2 polypeptide-expressing cell, or that are useful for treating or inhibiting the development of an ACE2-interacting coronavirus infection. Thus in certain aspects, the present disclosure relates to the use of a peptide that comprises, consists or consists essentially of the amino acid sequence set forth in SEQ ID NO: 1 or 2, or an amino acid sequence corresponding thereto (also referred to herein as “RBD mimetic peptides”) to identify compounds (agents) which are antagonists of an ACE2-interacting coronavirus:ACE2 polypeptide interaction/complex. Compounds identified through this screening can be tested to assess their ability to antagonize the binding of the ACE2-interacting coronavirus to an ACE2 polypeptide-expressing cell and/or inhibit entry of the ACE2-interacting coronavirus into an ACE2 polypeptide-expressing cell. Optionally, these compounds can further be tested in animal models to assess their ability to inhibit ACE2-interacting coronavirus infection.

There are numerous approaches to screening for therapeutic agents for inhibiting ACE2-interacting coronavirus infection by targeting the RBD mimetic peptide:ACE2 polypeptide interaction/complex. In certain embodiments, high-throughput screening of compounds can be carried out to identify agents that perturb this interaction. In certain embodiments, the assay is carried out to screen and identify compounds that specifically inhibit or reduce binding of an RBD mimetic peptide to an ACE2 polypeptide or ACE2 polypeptide-expressing cell. In a further embodiment, the compounds can be identified by their ability to interact with an RBD mimetic peptide or to an ACE2 polypeptide.

A variety of assay formats will suffice and, in light of the present disclosure, those not expressly described herein will nevertheless be comprehended by one of ordinary skill in the art. As described herein, the test or ‘candidate’ compounds (agents) of the invention may be created by any combinatorial chemical method. Alternatively, the compounds may be naturally occurring molecules that are extracted and purified from a suitable source, or synthesized in vivo or in vitro. Compounds (agents) to be tested can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In a specific embodiment, the test agent is a small organic molecule having a molecular weight of less than about 2,000 Daltons.

The test compounds can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of a candidate drug on the molecular target as may be manifest in an alteration of binding affinity between an RBD mimetic peptide and an ACE2 polypeptide or ACE2 polypeptide-expressing cell.

Candidate compounds may also be selected by electronic screening of well-known large compound libraries, such as the Available Chemical Directory (ACD; http://www.organicworldwide.net/content/available-chemical-directory). Compounds of such libraries may be analyzed by docking programs. In particular, to evaluate the quality of fit and strength of interactions between ligands or potential ligands and RBD mimetic peptide or ACE2 polypeptide binding sites, docking programs such as Autodock (available from Oxford Molecular, Oxford, UK), Dock (available from Molecular Design Institute, University of California San Francisco, Calif.), Gold (available from Cambridge Crystallographic Data Centre, Cambridge, UK) and FlexX and FlexiDock (both available from Tripos, St. Louis, Mo.) may be used. These programs and the program Affinity (available from Molecular Simulations, San Diego, Calif.) may also be used in further development and optimization of candidate compounds. Standard molecular mechanics force fields such as CHARMm and AMBER may be used in energy minimization and molecular dynamics.

Merely to illustrate, in an exemplary screening assay of the present disclosure, the compound of interest is contacted with an RBD mimetic peptide which is ordinarily capable of binding to an ACE2 polypeptide or ACE2 polypeptide-expressing cell.

In embodiments which employ an ACE2 polypeptide, the polypeptide may comprise the mature ACE2 amino acid sequence set forth for example in GenBank Accession: NP_001358344. The polypeptide may be conveniently prepared by recombinant techniques. For example, the ACE2 polypeptide may be prepared by a procedure including the steps of: (a) preparing a construct comprising a coding sequence for the ACE2 polypeptide, wherein the coding sequence is operably connected to a promoter; (b) introducing the construct into a host cell in which the promoter is operable; (c) culturing the host cell to express the coding sequence to thereby produce the encoded polypeptide; and (d) isolating the encoded polypeptide from the host cell. A representative ACE2 polypeptide coding sequence is set out in GenBank Accession NM_001371415, which may be used to design coding sequences for expressing a selected ACE2 polypeptide.

Recombinant polypeptides can be conveniently prepared using standard protocols as described for example in Ausubel et al. “Current Protocols in Molecular Biology”, John Wiley & Sons Inc, 1994-2003.

To the mixture of the compound and RBD mimetic peptide is then added a composition containing an ACE2 polypeptide. Detection and quantification of RBD mimetic peptide/ACE2 polypeptide complexes provides a means for determining the compound's efficacy at inhibiting complex formation between the RBD mimetic peptide and the ACE2 polypeptide. The efficacy of the compound can be assessed by generating dose response curves from data obtained using various concentrations of the test compound. Moreover, a control assay can also be performed to provide a baseline for comparison. For example, in a control assay, an isolated and/or purified ACE2 polypeptide is added to a composition containing an isolated and/or purified RBD mimetic peptide, and the formation of RBD mimetic peptide/ACE2 polypeptide complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

The capacity of a compound to modulate the interaction between the RBD mimetic peptide and an ACE2 polypeptide may be tested by any method which is known to the person of skills in the art to be suitable for assessing the interaction between two proteins. These methods include such technique as e.g., immunoblotting, immunoprecipitation analyses, fluorescence polarization, FRET (Fluorescence Resonance Energy Transfer), BRET (Bioluminescence Resonance Energy Transfer), AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIAcore™), isothermal titration calorimetry (ITC), differential scanning calorimetry, microscale thermophoresis, gel electrophoresis, and chromatography including gel filtration. These and other methods may take advantage of some fusion partner or label of the RBD mimetic peptide and/or ACE2 polypeptide. Assays may employ a variety of detection methods including but not limited to chromogenic, fluorescent, luminescent, or isotopic labels.

The present disclosure also contemplates the use of an interaction trap assay, also known as the “two hybrid assay”, for identifying agents that disrupt interaction between an RBD mimetic peptide and an ACE2 polypeptide. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present invention contemplates the use of reverse two hybrid systems to identify compounds (e.g., small molecules or peptides) that dissociate interactions between an RBD mimetic peptide and an ACE2 polypeptide. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

In order that the disclosure may be readily understood and put into practical effect, particular preferred embodiments will now be described by way of the following non-limiting examples.

### EXAMPLES

### Example 1

### Screening Drug Libraries to Identify Drugs that Bind ACE2 and Screening for a Subset of Drugs that Block ACE2 SARS-CoV2 Interaction with Surface Plasmon Resonance

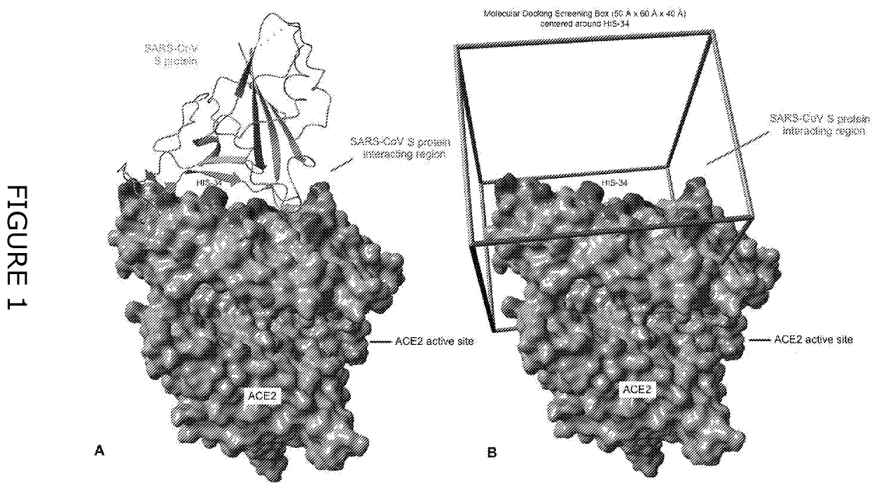

Two peptides (RBD-mimic1: NCYFPLQSYGFQPTNGV [SEQ ID NO: 1] and RBD-mimic2: NCYFPLQSYGFQPTNGVGY [SEQ ID NO: 2]) were designed and synthesized containing key ACE2-interacting residues of the SARS-CoV-2 spike protein RBD, based on the model SARS-CoV-2 spike protein RBD-ACE2 complex (PDB 6VW1; see FIG. 1a). SPR studies showed that RBD-mimic 1 binds recombinant human ACE2 with high affinity (K_{D}=13.7±2.4 nM; FIG. 1b), which essentially recapitulates the recently reported ˜15 nM K_{D} for the SARS-CoV-2 Spike protein-ACE2 interaction (Hoffmann et al., Cell 2020, 181(2):271-28). RBD-mimic2 was shown to have a lower affinity (K_{D} of 347.2±102 nM).

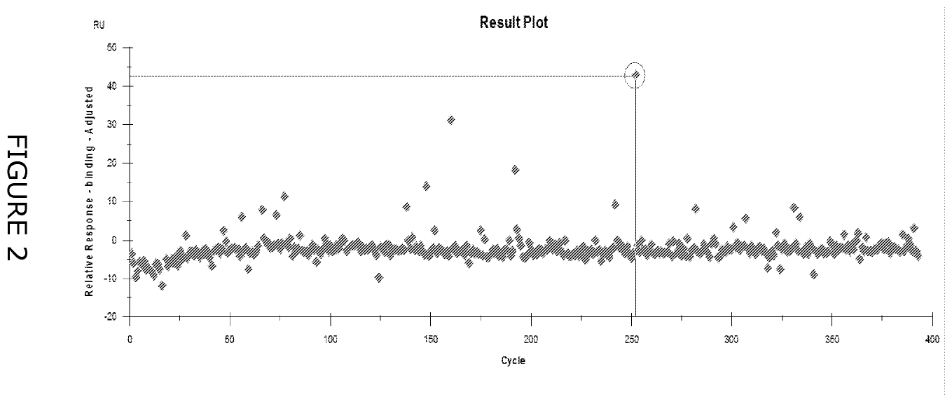

Recombinant human ACE2 protein was tested for binding to 3141 FDA approved compounds with Surface Plasmon Resonance (SPR). Initial screening of the compounds at 1 μM identified 5 compounds that bound with high affinity (see, FIG. 2 which illustrates identification of Evans Blue).

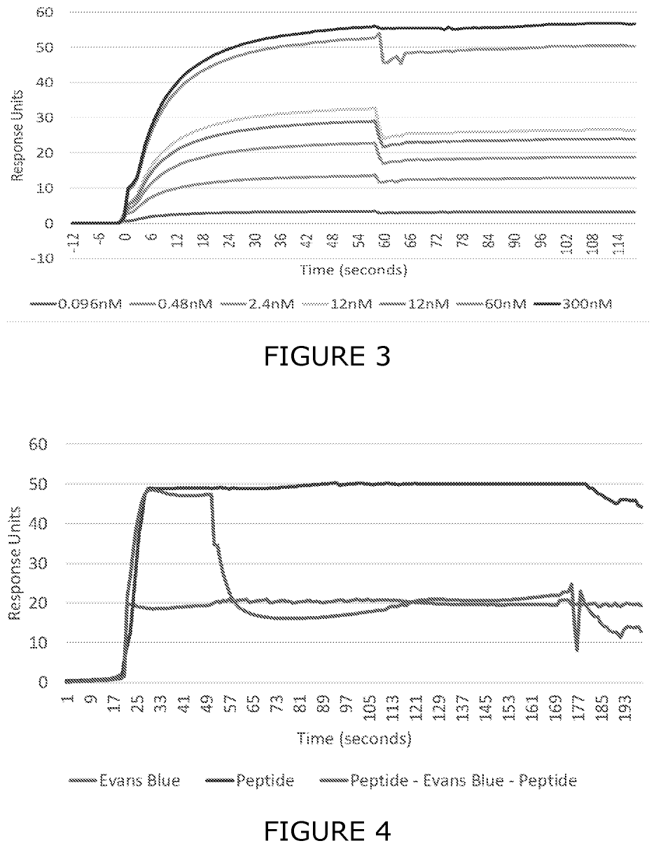

These 5 compounds were rescreened for binding affinity and ability to block SARS-CoV2 spike protein derived peptide, RBD-mimic1, from binding to human ACE2 in direct competition SPR experiments (see, FIG. 3 which illustrate surface plasmon resonance curves for Evans blue). Only 1 of the 6 compounds bound with high affinity and were able to completely block interactions with SARS-CoV2 spike protein derived peptide (RBD-mimic1 peptide). The drug shown in Table 4 can block interactions between ACE2 and RBD-mimic1 peptide.

TABLE 4
COMPOUNDS FROM DRUG SCREEN WITH AFFINITY
BELOW 1 μM THAT ARE PREDICTED TO BLOCK
SARS-CoV2 INTERACTIONS WITH HUMAN ACE2
IN SURFACE PLASMON RESONANCE EXPERIMENTS.

| | | 1:1 Competition between compound |
|---|---|---|
| Compound | Kd [nM] | vs RBD-mimic1 peptide @ 1 μM |
| Evans Blue | 1.63 ± 0.08 | Yes (99.8% ± 3.84) |

The data in Table 4 show that in 1:1 competition Evans blue can ˜100% block ACE2 binding to RBD-mimic1 peptide. Evans blue and RBD-mimic1 peptide were flowed at 1 μM (see FIG. 3).

### Materials and Methods

### Reagents

Purified ACE2 protein was sourced from R&D Systems and Assay Matrix.

RBD-mimic1 peptide was designed based on the interacting sequence in the models and was produced by Mimotopes.

### Drug Libraries

A combination of two libraries, Microsource-CPOZ (2400 drugs) and ML Drug (741 drugs) libraries, including FDA approved drugs, were purchased from Compounds Australia (Compounds Australia; Griffith Institute for Drug Discovery, Griffith University, Building N75, Brisbane Innovation Park, Don Young Road, Nathan QLD 4111).

### Drugs for Post-Screen Assays

All drugs for secondary SPR competition screening were obtained from SIGMA Aldrich (St Louis, Missouri): Evans Blue E2129.

### Methods

### Surface Plasmon Resonance (SPR)

SPR analyses were performed using a Biacore S200 System (GE Healthcare Life Sciences, Parramatta, NSW AUS). Samples were analyzed at 25° C. in phosphate buffered saline (PBS), at a flow rate of 10 μL/min, and by using single cycle kinetics. Human ACE2 proteins from different sources were immobilized onto separate cells of a Series S CM5 sensor chip using a NHS capture kit (both from GE Healthcare Life Sciences). A blank immobilization was used as a control/reference (flow cell 1) on all chips. Affinity data points were taken at 15 s after injection to avoid an artefactual signal resulting from bulk transport. SPR sensorgrams were analyzed using Biacore Evaluation software (GE Healthcare Life Sciences) to determine dissociation constants (i. e., K_{D}).

### Repurposed Drug Screen Against Human ACE2

Human ACE2 was immobilized onto a CM5 sensor chip with a blank control flow cell as described above. A combination of two libraries, including FDA approved drugs, Microsource-CPOZ (2400 drugs) and ML Drug (741 drugs), were purchased from Compounds Australia. Each drug was made up to 1 μM in 10% DMSO in a 384 well plate just prior to use in the Biacore S200 system. A new biosensor chip was made for each 384 well plate screened. A single concentration injection screen (yes/no) binding assay was performed. Binding was determined based on a response unit shift equal to the molecular weight corrected response units of the positive control glycan on the stability of binding phase of the dissociation cycle. “Hits” were rescreened across a concentration range of 1.6 nM to 1 μM to define the K_{D} of the interaction. Any drug with a K_{D} value >1 μM was discarded from the analysis. K_{D} assays to determine high affinity interactions were further refined by using lower concentration ranges of compound to get the final affinity, as described in figure legends.

To determine if drugs with high affinity interactions with the human ACE2 could inhibit the interaction with RBD-mimic1 peptide, competition assay between ACE2 and compounds/peptide was performed as described Mubaiwa et al. (2017, Sci Rep. 7(1):5693). Peptide, drug or drug and peptide were flowed at 1 μM over the human ACE2 proteins and the response units of the interactions were recorded. All SPR sensorgrams and results plots were analyzed with Biacore S200 Evaluation Software (GE Healthcare Life Sciences).

### Example 2

### Screening Drug Libraries to Identify Drugs that Bind ACE2 and Screening for a Subset of Drugs that Block ACE2 SARS-CoV2 Interaction with Molecular Docking

A three-dimensional x-ray crystal structure of human ACE2 was obtained from the protein databank (http://rcsb.org) using the structure of SARS coronavirus spike RBD complexed with the human ACE2 receptor (PDB 2AJF). A rectangular box with dimensions 50 Å×60 Å×40 Å (x, y, and z) was centered at the amino acid HIS-34 of the ACE2 coordinates (see FIG. 1). A total of 57,641 compounds were docked against human ACE2 with a total computing time of ˜7 days. This virtual molecular docking screen identified 14 compounds that bound to the boxed region of the human ACE2 protein.

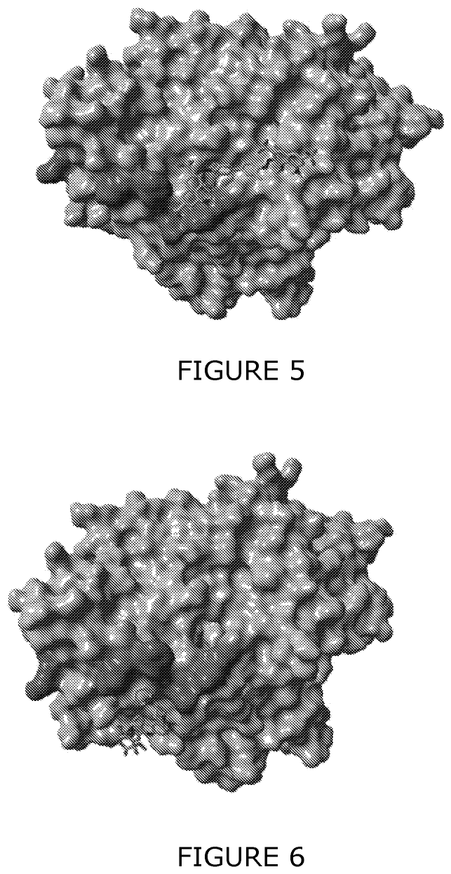

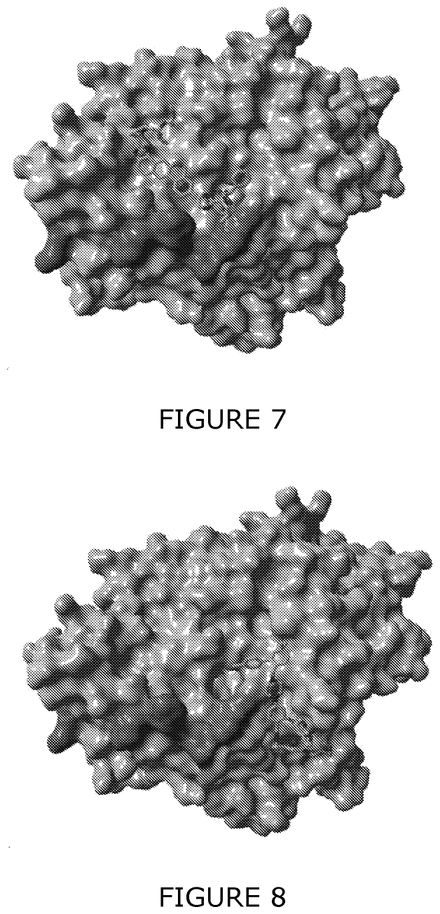

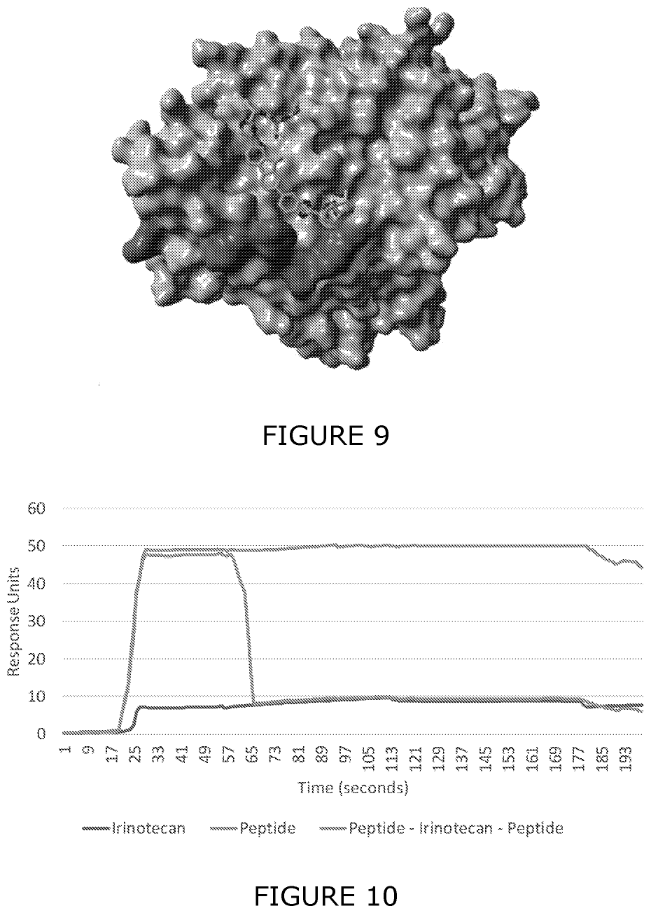

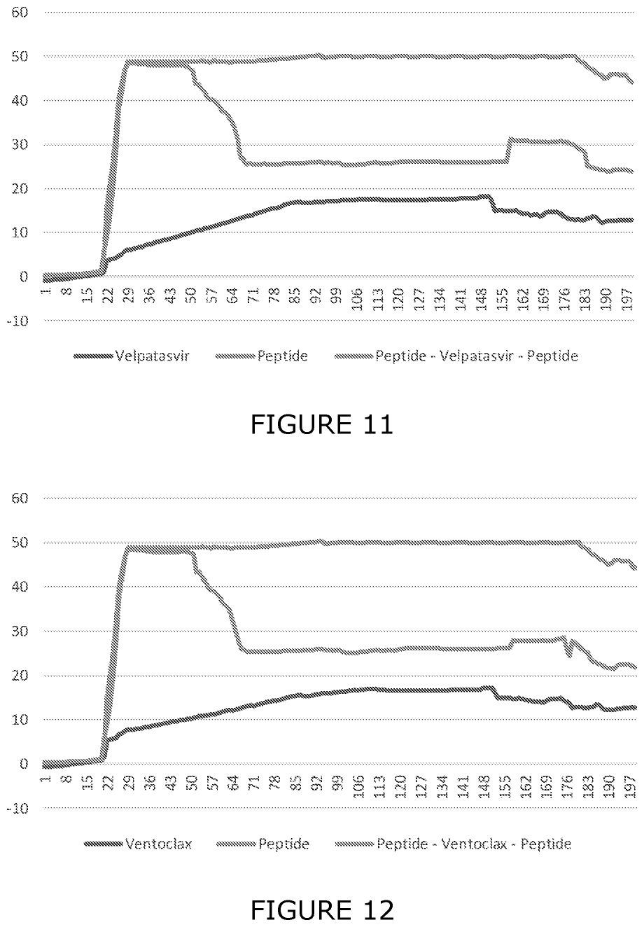

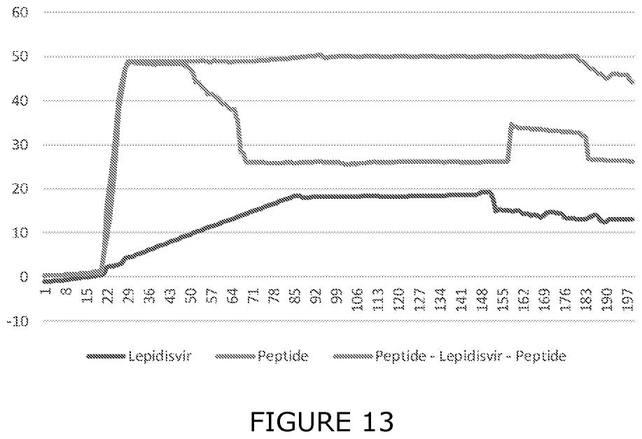

Only 9 of the 14 identified compounds could be sourced for a secondary screening using surface plasmon resonance (SPR) to test for binding affinity and the ability to block RBD-mimic1 peptide binding to human ACE2 in direct competition SPR experiments (see, FIGS. 5-9 which illustrate the molecular docking analysis of Evans blue, Irinotecan, Velpatasvir, Venetoclax, Ledipasvir, respectively). Only 2 of the 9 compounds (Irinotecan and Evans Blue) bound not only with high affinity to ACE2 but were able to completely block interactions with the RBD-mimic1 peptide (see FIGS. 4 and 10 respectively). Additionally 3 other compounds could block more than 60% of the ACE2:RBD-mimic1 peptide interaction (Velpatasvir, Venetoclax, Ledipasvir, see FIG. 11-13).

Tables 5 shows the relative activity of the 5 compounds in inhibiting binding of human ACE2 to SARS-CoV2 derived peptide. Drug or drug and RBD-mimic1 peptide were flowed at 1 μM.

TABLE 5
COMPOUNDS FROM MOLECULAR DOCKING DRUG
SCREEN THAT COULD BLOCK SARS-CoV2 INTERACTIONS
WITH HUMAN ACE2 IN SURFACE PLASMON
RESONANCE EXPERIMENTS.

| | | 1:1 Competition between compound | |
|---|---|---|---|
| NAME | Kd [nM] | vs RBD-mimic1 peptide @ 1 μM | |
| Evans Blue | 1.63 ± 0.08 | Yes | (99.8% ± 3.84) |
| Velpatasvir | 24.9 ± 4.24 | Partial | (61.4% ± 0.34) |
| Venetoclax | 290.6 ± 31.1 | Partial | (66.3% ± 4.1) |
| Ledipasvir | 417.2 ± 50.7 | Partial | (79.4% ± 2.1) |
| Irinotecan | 825.2 ± 112 | Yes | (100% ± 6.36) |

### Materials and Methods

### Reagents

Purified ACE2 protein was sourced from R&D Systems and Assay Matrix.

RBD-mimic1 peptide was designed based on the interacting sequence in the models and was produced by Mimotopes.

### Drugs for Post-Screen Assays

Drugs for competition and other SPR analysis were obtained from SIGMA Aldrich (St Louis, Missouri): Evans blue E2129 and SelleckChem (Houston, Texas): Velpatasvir Cat #S3724, Ledipasvir Cat #S7579, Compounds Australia: Venetoclax, Irinotecan.

### Methods

### Molecular Docking Screens

Accession of target protein and box selection—The three-dimensional structure of human ACE2 was obtained from the protein databank (http://rcsb.org) using the structure of SARS coronavirus spike RBD complexed with the human ACE2 receptor (PDB 2AJF) 6 with 2.9 Å resolution. A rectangular box with dimensions 50 Å×60 Å×40 Å (x, y, and z) was centered at the amino acid HIS-34 of the ACE2 coordinates as seen in FIG. 1. The human ACE2 structure was cleaned by deleting the SARS coronavirus spike protein and all water molecules. Subsequent to our screen the region of SARS-CoV-2-ACE2 interaction was modelled 7, and is consistent with a recent cryo-EM study of the SARS-CoV-2-ACE2 complex at 3.6 Å 4, showing that SARS-Cov-2 and SARS-CoV interact with a similar region of ACE2 i.e. that region boxed in FIG. 1.

Ligand selection—Chemical structures of ligands were downloaded from multiple libraries (Approved Drugs—4,195, Charitee Super drugs—1,050, eDrugs—1,610, Ligandbox Kegg—5,814, PRESTWICK OFF PATENTS—2,062, OTAVA—9,765, CHEMDIV—33,145) as 2D SDF molecular format. 3D conformers of all ligands are needed for molecular docking screening and were generated using DataWarrior (version 4.7.2) software utilizing the MMFF94s+ forcefield 8. A total of 57,641 compounds were docked against human ACE2 with a total computing time of ˜7 days using the Griffith University high performance computing cluster and two Windows workstations.

Target and ligand optimization—Molecular screening of the molecular database was performed using Autodock VINA 9 that is implemented in the YASARA software suite 10. The macro dock_runscreening.mcr was used and modified to dock the molecular library to human ACE2 (PDB 2AJF) using 12 docking runs per ligand in a completely flexible mode with an average time requirement of 12 seconds per ligand using Griffith University high performance cluster and two Windows Workstations.

SPR analyses were performed using a Biacore S200 System (GE Healthcare Life Sciences, Parramatta, NSW AUS). Samples were analyzed at 25° C. in phosphate buffered saline (PBS), at a flow rate of 10 μL/min, and by using single cycle kinetics. Human ACE2 proteins from different sources were immobilized onto separate cells of a Series S CM5 sensor chip using a NHS capture kit (both from GE Healthcare Life Sciences). A blank immobilization was used as a control/reference (flow cell 1) on all chips. Affinity data points were taken at 15 s after injection to avoid an artefactual signal resulting from bulk transport. SPR sensorgrams were analyzed using Biacore Evaluation software (GE Healthcare Life Sciences) to determine dissociation constants (i.e., K_{D}).

### Surface Plasmon Resonance (SPR)

SPR analyses were performed using a Biacore S200 System (GE Healthcare Life Sciences, Parramatta, NSW AUS). Samples were analyzed at 25° C. in phosphate buffered saline (PBS), at a flow rate of 10 μL/min, and by using single cycle kinetics. Human ACE2 proteins from different sources were immobilized onto separate cells of a Series S CM5 sensor chip using a NHS capture kit (both from GE Healthcare Life Sciences). A blank immobilization was used as a control/reference (flow cell 1) on all chips. Affinity data points were taken at 15 s after injection to avoid an artefactual signal resulting from bulk transport. SPR sensorgrams were analyzed using Biacore Evaluation software (GE Healthcare Life Sciences) to determine dissociation constants (i. e., K_{D}).

### Repurposed Drug Screen Against Human ACE2

Human ACE2 was immobilized onto a CM5 sensor chip with a blank control flow cell as described above. A combination of two libraries, including FDA approved drugs, Microsource-CPOZ (2400 drugs) and ML Drug (741 drugs), were purchased from Compounds Australia. Each drug was made up to 1 μM in 10% DMSO in a 384 well plate just prior to use in the Biacore S200 system. A new biosensor chip was made for each 384 well plate screened. A single concentration injection screen (yes/no) binding assay was performed. Binding was determined based on a response unit shift equal to the molecular weight corrected response units of the positive control glycan on the stability of binding phase of the dissociation cycle. “Hits” were rescreened across a concentration range of 1.6 nM to 1 μM to define the K_{D} of the interaction. Any drug with a K_{D} value >1 μM was discarded from the analysis. K_{D} assays to determine high affinity interactions were further refined by using lower concentration ranges of compound to get the final affinity, as described in figure legends.

To determine if drugs with high affinity interactions with the human ACE2 could inhibit the interaction with RBD-mimic1 peptide, competition assay between ACE2 and compounds/peptide was performed as described Mubaiwa et al. (2017, Sci Rep. 7(1):5693). Peptide, drug or drug and peptide were flowed at 1 μM over the human ACE2 proteins and the response units of the interactions were recorded. All SPR sensorgrams and results plots were analyzed with Biacore S200 Evaluation Software (GE Healthcare Life Sciences).

### Example 3

### Structure Affinity Relationship Studies of Non-Labile Linker of Prodrug Irinotecan

Irinotecan is an anti-cancer (antineoplastic or cytotoxic) chemotherapeutic drug. It is a carbamate prodrug and was designed to deliver camptothecin as a predominant topoisomerase I inhibitor for anticancer therapy.

Irinotecan hydrochloride salt 210 (CPT-11, Camptosar; Pfizer) is a parenteral aqueous soluble carbamate prodrug of antineoplastic topoisomerase I SN-38 (7-ethyl-10-hydroxy-camptothecin). In Irinotecan, a dipiperidino ionizable pro-moiety is linked to the phenol functionality of SN-38 by a carbamate bond, thus improving the overall aqueous solubility. The bioconversion of Irinotecan back to SN-38 occurs primarily by human liver microsomal carboxylesterases, CES 1A1 and CES2, which release the piperidinopiperidine moiety and SN-38, the active form of the drug.

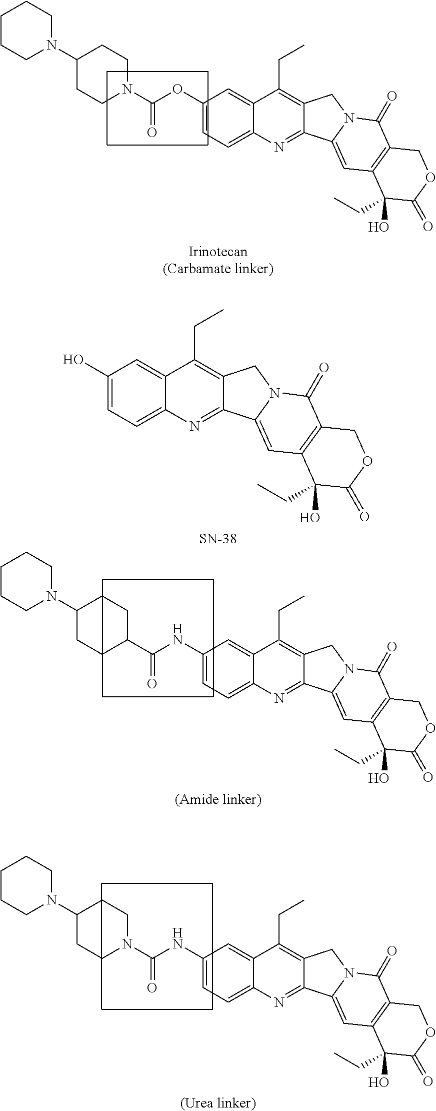

Irinotecan has been found to bind effectively to ACE2 (KD 825.2±112 nM, and a complete ability to block RBD-mimic1 peptide binding to human ACE2 in direct competition SPR experiments. The dipiperidino ionizable pro-moiety of Irinotecan has been found to be essential in interacting with ACE2. Therefore, it is proposed herein to replace the labile carbamate linker with an amide or urea linker.

TABLE 6
Structure Affinity Relationship (SAR) studies of SN-38, Irinotecan
and Irinotecan and two linker modifications (Amide and Urea
linker). Irinotecan, with a liable carbamate linker has a 9.66
times better binding affinity than the SN-38. However, Irinotecan
derivatives with an Amide or Urea linker have an 16.43 and
29.95 times better binding affinity than SN-38.

| NAME | Relative KDs |
|---|---|
| SN-38 | 1.00 |
| Irinotecan | 9.66 |
| Irinotecan (Amide linker) | 16.43 |
| Irinotecan (Urea linker) | 29.95 |

### Material and Methods

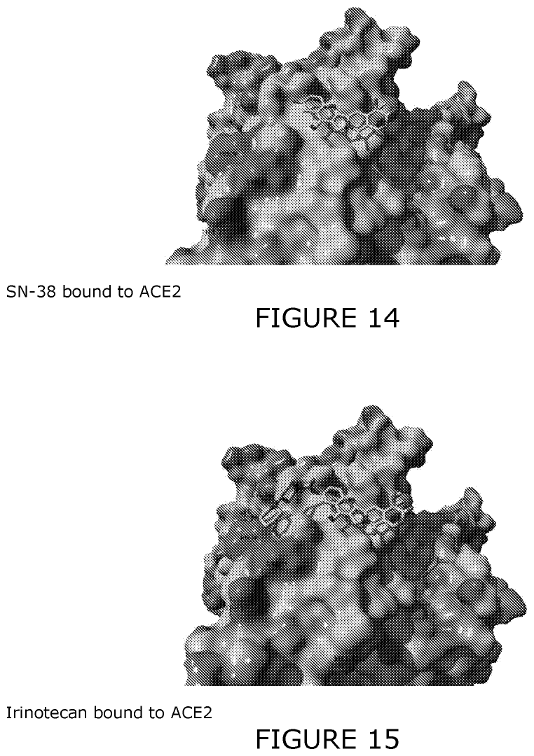

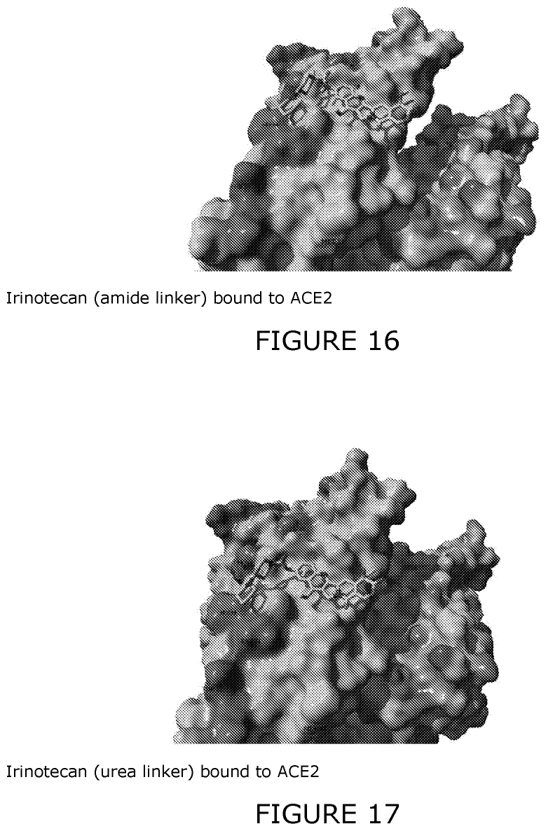

For SAR studies and the selection of the right non-labile linker the structure of Irinotecan as identified in the molecular docking screening was used to re-score the compounds without linker (SN-38) and with the linker modifications (Amide and Urea). The molecules were generated, energy optimized and a cubic box with dimensions 5 Å×5 Å×5 Å (x, y, and z) was centered at compound using complex coordinates. A local docking and re-scoring function was employed and relative affinities values are reported in Table 6. All docked and re-scored Irinotecan-related compounds are shown FIGS. 14 to 17.

### Example 4

### Inhibition of SARS-CoV-2 In Vitro Infection of Vero-E6 Cells by the Inhibitors Identified in the Drug Screens Described Herein

In vitro infection of Vero-E6 cells by SARS-CoV-2 is a frequently used model system in SARS-CoV-2, however it has limitations in common with all immortalized cell lines. When the compounds listed in Table 5 were tested in this assay, toxicity was observed with Velpatasvir, Venetoclax, Ledipasvir and Irinotecan that prevented their assessment for inhibition of SARS-CoV-2 infection (Day et al. 2021 mBio 12:e03681-20). In some cases, the Vero cell toxicity is evident at concentrations well below the known human therapeutic Cₘₐₓ (maximum concentration of drug in serum) of the same drugs in clinical use and these data highlight the limitations of this assay for evaluation of candidate drugs and as an initial screening tool (Day et al. 2021 mBio 12:e03681-20). Given the limitations of the Vero-E6 model, the identified compounds listed in Table 5 that could not be assessed in this assay remain as viable candidates for repurposing to treat SAR-CoV-2 infection.

Evans Blue displayed low toxicity and could be assessed in the Vero-E6 SARS-CoV-2 in vitro infection assay. Table 7 shows the activity of Evans blue (IC₅₀ 28.1±1.2 μM) in comparison to a known inhibitor of SARS-CoV-2 infection, Suramin (IC₅₀ 28.1±1.2 μM).

TABLE 7
IN VITRO POTENCY OF HITS AGAINST SARS-COV-2 INFECTION
AND CYTOTOXICITY TOWARD VERO-E6 CELLS.

| | IC₅₀ (μM) | | CC₅₀ (μM) | | |
|---|---|---|---|---|---|
| NAME | Mean ± SD | n^{a} | Mean ± SD | n^{a} | SI^{b} |
| Evans Blue | 28.1 ± 1.2 | 3 | >1,000 | 3 | >35.6 |
| Suramin | 28.1 ± 1.2 | 3 | >1,000 | 3 | >21.6 |

Vero-E6 cells were incubated with virus and compound for 24 h (50% inhibitory concentration [IC50] and 50% cytotoxic concentration [CC50] data) to 48 h at 37° C. Infection was measured with in situ ELISA, and IC50 values were determined by nonlinear regression of dose-response curves. The CC50 values were determined with an alamarBlue assay in identical experimental conditions (Day et al. 2021 mBio 12:e03681-20). ^{a} number of independent replicate studies, ^{b} SI, selectivity index: SI=CC50/IC50.

### Material and Methods

Cells and virus. Vero-E6 cells were maintained in advanced minimal essential medium (MEM) supplemented with 5% fetal bovine serum (FBS) at 37° C. in a humidified atmosphere of 5% CO2. SARS-CoV-2 strain SARS-CoV-2-CoV-2/Australia/QLD02/2020 (GISAID accession code EPI_ISL_407896) was obtained from the Forensic and Scientific Services Unit of Queensland Health, Australia. The virus was propagated in Vero-E6 cells in medium supplemented with 2% FBS (infection medium). All work involving live SARS-CoV-2 cultures was carried out in a certified physical containment level 3 (PC3) facility at the Institute for Glycomics, Griffith University.

Virus propagation and titration. SARS-CoV-2 stocks were prepared by infecting confluent Vero-E6 cells at a multiplicity of infection (MOI) of 0.05 for 72 h at 37° C. Infection supernatants were clarified by centrifugation at 4,000×g for 15 min, homogenized, aliquoted, and stored at −80° C. Virus stock titers were determined by focus-forming assays as follows: confluent Vero-E6 cells in 96-well plates were infected with 10-fold dilutions of virus in 50 μL for 1 h at 37° C., after which 50 μL of infection medium containing 1% Avicel (FMC BioPolymer) was added to each well. Plates were further incubated for 24 h at 37° C. SARS-CoV-2 foci were obtained by following the in situ enzyme-linked immunosorbent assay (ELISA) procedure detailed below, but by adding 50 μL per well of TrueBlue peroxidase substrate (KPL) in place of tetramethylbenzidine (TMB) reagent until dark blue foci appeared. Wells were subsequently rinsed with running water, and foci were manually counted to determine the focus-forming units (FFU) per ml.

Biological screening of drug candidates. Drug screening was done following similar methods previously described for SARS-CoV-2 (Holwerda et al. 2020. Microorganisms 8:1872) and another virus (Earley et al. 2019. ACS Infect Dis 5:1708-1717). Briefly, Vero-E6 cells were seeded in 96-well plates at a density of 1.75×104 cells per well. On the day of infection, the medium in each well was removed and replaced with the subsequent addition of 50 μL of infection medium, 25 μL of compound dilution in infection medium (30 min before infection), and 25 μL of SARS-CoV-2 dilution. The final volume in each well was 100 μL, and infection was done at an MOI of 0.002. Virus and compound mixtures were left in place, and cells were incubated for 48 h at 37° C. and 5% CO2 before infection was measured using in situ ELISA. Compounds were evaluated in technical duplicates.

Dose-response experiments. Assays were conducted as for the drug screenings, but Vero-E6 cells were infected at an MOI of 0.12 and infections carried out for 24 h to mitigate the risks of compound-induced cytotoxicity observed during longer incubation times. This MOI value was selected as it yielded a maximum signal falling within the upper linear range of an MOI-response curve measured with in situ ELISA. Infection in the presence of compound was measured with in situ ELISA, and compounds were evaluated in technical triplicates. The compound concentrations that inhibit 50% of SARS-CoV-2 infection (IC50 values) were determined by nonlinear regression of dose-response curves using GraphPad Prism 8.

In situ ELISA. ELISAs were adapted from previously published methods (60). Infected cells in 96-well plates were fixed by addition of 100 μL per well of an 8% paraformaldehyde solution in phosphate-buffered saline (PBS) for 30 min at room temperature. Cells were subsequently permeabilized and endogenous peroxidases inhibited with 1% IGEPAL and 0.3% H₂O₂ in PBS, respectively, for 20 min at 37° C. The intracellular SARS-CoV-2 nucleocapsid was immunostained by incubating cells with a 1:2,000 dilution of primary mouse anti-SARS-CoV-2 nucleocapsid antibody (reference no. 40143-MM08; SinoBiological) in PBS/5% skim-milk for 30 min at 37° C., and a 1:6,000 dilution of secondary goat anti-mouse IgG(H+L)-horseradish peroxidase (HRP)-conjugated antibody (ref. 170-6516; Bio-Rad) in PBS/5% skim-milk for 30 min at 37° C. The cell monolayers were washed three times for 5 min with PBS/0.02% Tween 20 after each of the aforementioned incubations. Nucleoprotein levels were detected using 50 μL per well of OptEIA TMB substrate (BD Biosciences), and the reactions stopped with 25 μL per well of 0.6 M H₂SO₄. The absorbance at 450 nm was read in each well using an X-Mark microplate absorbance spectrophotometer (Bio-Rad). Percentages of infection were calculated by subtracting the background absorbance of negative-control wells (non-infected cells) from all other wells and normalizing the resulting values to positive-control wells (infected cells, not treated).

Drug cytotoxicity assays. Compound dilutions were incubated with Vero-E6 cells in 96-well plates in the absence of virus, in infection medium, for 24 h at 37° C. They were subsequently discarded, and the cell monolayers were washed twice with 100 μL of infection medium before applying 50 μL per well of 10% alamarBlue (Thermo Fisher) in serum-free advanced MEM. Plates were further incubated for 2 to 4 h at 37° C., and absorbances were read in each well at 570 nm and 600 nm using an X-Mark microplate absorbance spectrophotometer (Bio-Rad). Cellular viability was calculated following the manufacturer's instructions and expressed as the percentage of control (untreated cells). The compound concentrations inducing 50% cytotoxicity (CC₅₀ values) were determined by nonlinear regression of dose-response curves using the software GraphPad Prism 8. Compounds were evaluated in technical triplicates.

The disclosure of every patent, patent application, and publication cited herein is hereby incorporated by reference herein in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1.-33. (canceled)

34. A method of inhibiting interaction of a coronavirus with an ACE2 polypeptide-expressing cell in a subject, the method comprising administering to the subject an ACE2 polypeptide-interacting compound that inhibits binding of an ACE2-interacting coronavirus spike protein RBD structural mimetic peptide comprising the amino acid sequence set forth in SEQ ID NO: 1 or 2 to the ACE2 polypeptide, wherein the an ACE2 polypeptide-expressing cell is contacted with the ACE2 polypeptide-interacting compound, and wherein the ACE2 polypeptide-interacting compound is Irinotecan, an Irinotecan derivative or a pharmaceutically acceptable salt thereof.

35. The method of claim 34, wherein the Irinotecan derivative is a compound represented by formula (IIIb): 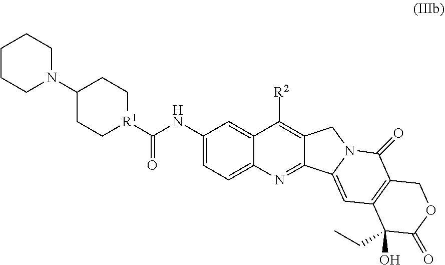
wherein:
R¹ is selected from carbon and nitrogen; and
R² is selected from hydrogen and C₁₋₃ alkyl,
or a pharmaceutically acceptable salt thereof.

36. The method of claim 35, wherein the Irinotecan derivative is a compound selected from the group consisting of:
a compound of formula IIIb1: 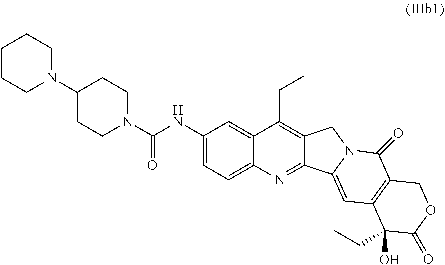 [SMILES: CCc1c2c(nc3ccc(NC(=O)N4CCC(N5CCCCC5)CC4)cc13)-c1cc3c(c(=O)n1C2)COC(=O)[C@]3(O)CC]
or a pharmaceutically acceptable salt thereof,
a compound of formula IIIb2: 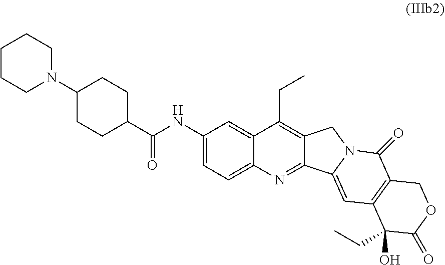 [SMILES: CCc1c2c(nc3ccc(NC(=O)C4CCC(N5CCCCC5)CC4)cc13)-c1cc3c(c(=O)n1C2)COC(=O)[C@]3(O)CC]
or a pharmaceutically acceptable salt thereof;
a compound of formula IIIb3: 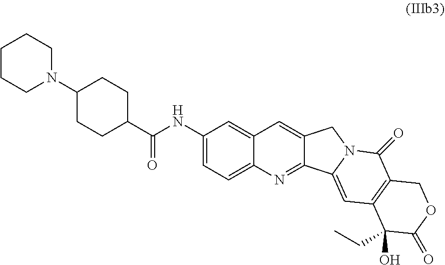 [SMILES: CC[C@@]1(O)C(=O)OCc2c1cc1n(c2=O)Cc2cc3cc(NC(=O)C4CCC(N5CCCCC5)CC4)ccc3nc2-1]
or a pharmaceutically acceptable salt thereof; and
a compound of formula IIIb4: 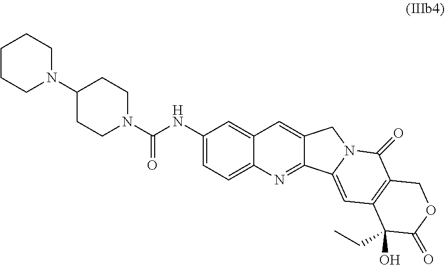 [SMILES: CC[C@@]1(O)C(=O)OCc2c1cc1n(c2=O)Cc2cc3cc(NC(=O)N4CCC(N5CCCCC5)CC4)ccc3nc2-1]
or a pharmaceutically acceptable salt thereof.

37. The method of claim 34, wherein the interaction is one or both of binding of the coronavirus to the cell and entry of the coronavirus into the cell.

38. The method of claim 34, wherein the cell is a lung cell, an enterocyte, an endothelial cell, an epithelial cell, a kidney cell, or an arterial smooth muscle cell.

39. The method of claim 34, wherein the cell is a cell of the respiratory tract.

40. The method of claim 34, wherein the coronavirus is Severe Acute Respiratory Syndrome virus 2 (SARS-CoV-2).

41. The method of claim 34, comprising systemically administering the ACE2 polypeptide-interacting compound to the subject.

42. The method of claim 34, comprising topically administering the ACE2 polypeptide-interacting compound to the subject.

43. The method of claim 34, comprising orally administering the ACE2 polypeptide-interacting compound to the subject.

44. The method of claim 34, comprising intranasally or inhalationally administering the ACE2 polypeptide-interacting compound to the subject.

45. The method of claim 34, comprising administering the ACE2 polypeptide-interacting compound to the respiratory tract of the subject.

46. The method of claim 34, comprising concurrently administering to the subject an ancillary agent with the ACE2 polypeptide-interacting compound.

47. A peptide compound that consists or consists essentially of the amino acid sequence set forth in SEQ ID NO: 1 or 2, or an amino acid sequence corresponding thereto.

48. A method of eliciting an immune response to an ACE2-interacting coronavirus in a subject, the method comprising immunizing the subject with a peptide that consists or consists essentially of the amino acid sequence set forth in SEQ ID NO: 1 or 2, or an amino acid sequence corresponding thereto, to thereby elicit an immune response to the ACE2-interacting coronavirus in the subject.
